(19) United States
(12) Patent Application Publication
(10) Pub. No.: US 2025/0352629 A1
(43) Pub. Date: Nov. 20, 2025

(54) ADJUVANT FOR BACILLUS CALMETTE-GUERIN CANCER IMMUNOTHERAPY

(71) Applicant: The Royal Institution for the Advancement of Learning/McGill University, Montréal (CA)

(72) Inventors: Leonardo F. JURADO, Montréal (CA); Maziar Divangahi, Montréal (CA)

(21) Appl. No.: 19/209,068

(22) Filed: May 15, 2025

(30) Foreign Application Priority Data
May 17, 2024 (CA) 3239012

(51) Int. Cl. A61K 39/04; A61K 39/00; A61K 39/39; A61P 35/00

(52) U.S. Cl. CPC A61K 39/04; A61K 39/39; A61P 35/00; A61K 2039/522; A61K 2039/55583

(57) ABSTRACT

There is provided the combination of Bacillus Calmette-Guérin (BCG) vaccine and β-glucan adjuvant for the treatment of a cancer characterized by the presence of protumoral T3 neutrophils in the tumour microenvironment. The β-glucan is characterized by a μ-1,3 glucose backbone. The BCG and β-glucan combination demonstrated a synergistic effect in remodeling the tumour microenvironment to resist conversion of neutrophils into the T3 phenotype. The cancer can be bladder cancer, melanoma, lung adenocarcinoma, head and neck squamous cell cancer, pancreatic adenocarcinoma, low-grade gliomas, esophageal carcinoma, and cervical squamous cell carcinoma.

### CROSS-REFERENCE TO A RELATED APPLICATION

This disclosure claims priority from Canadian patent application number 3,239,012 filed on May 17, 2024, which is incorporated herein by reference in its entirety.

### TECHNICAL FIELD

This disclosure relates to the field of solid cancer treatments, more specifically immunotherapy treatment of cancer such as bladder cancer and melanoma.

### BACKGROUND OF THE ART

Cancer is the second leading cause of death globally. Bladder cancer (BC) is the ninth most common malignancy worldwide and the fifth most prevalent in both Europe and the United States. Bacillus Calmette-Guérin (BCG) intravesical immunotherapy is the established treatment for BC, particularly for high-risk non-muscular invasive BC which accounts for 80% of the cases. While BCG was initially developed as a vaccine against tuberculosis, it has transitioned into the gold-standard immunotherapy for BC.

Unfortunately, although BCG immunotherapy induces a complete response rate in a significant proportion of patients with high-risk tumors, 40 to 60% of patients experience recurrence within five years. In addition, the effectiveness of BCG immunotherapy depends on repetitive treatment (at least 12 doses during the first 6 months) with a high dose of BCG (1×10⁸ CFU), which is associated with several adverse effects, leading to intolerance in about 20% of cases and treatment adjustment. Furthermore, for non-responders, radical cystectomy is recommended, severely compromising patient's quality of life. Thus, enhancing the effectiveness of BCG therapy, including a decreased need for repeated treatment, is highly desirable, to offer significant benefits to individuals battling bladder cancer.

Melanoma is a malignancy that originates in melanocytes in the skin. Although there are many types of skin cancers, melanoma is responsible for the majority of deaths. In most cases, melanoma metastases are already present at the time of diagnosis. Metastatic cancers are heterogenous and it is desired to treat such heterogenous diseases with multiple drugs having different mechanism of actions. Accordingly, it would be beneficial to be provided with additional drugs to treat melanoma as it is often the case that two or more different drugs are administered to treat melanoma and cancers more generally.

### SUMMARY

In one aspect, there is provided a method of treating cancer in a subject in need thereof with a therapeutically effective amount of Bacillus Calmette-Guérin (BCG) vaccine and β-glucan adjuvant. The cancer is characterized by the presence of protumoral T3 neutrophils in the tumour microenvironment, and is, for instance, bladder cancer, melanoma, lung adenocarcinoma, head and neck squamous cell cancer, pancreatic adenocarcinoma, low-grade gliomas, esophageal carcinoma, and cervical squamous cell carcinoma, preferably bladder cancer, more preferably non-muscular invasive bladder cancer.

The β-glucan has a β-1,3 glucose backbone, preferably. The β-glucan can also have β-1,6 side branching. The β-glucan is optionally derived from fungus or yeast. The β-glucan may have a molecular weight of from 100 kDa to 900 kDa.

The BCG vaccine can comprise an attenuated strain of Mycobacterium bovis which is a TICE strain, Frappier strain, Danish strain 1331, Glaxo 1077 strain, Tokyo 172-1 strain, Pasteur 1173 P2 strain, Moscow-I strain, RIVM strain, Connaught strain, Russia strain or Moreau strain.

Many further features and combinations thereof concerning the present improvements will appear to those skilled in the art following a reading of the instant disclosure.

### DESCRIPTION OF THE DRAWINGS

FIG. 1A is a schematic representation of the experimental design for the murine orthotopic bladder cancer model (MB49 tumor cell line) and intravesical (IB) BCG instillations.

FIG. 1B is a macroscopic image of a naïve mouse bladder.

FIG. 1C is a microscopy histologic image of a naïve mouse bladder.

FIG. 1D is a macroscopic image of a neoplastic mouse bladder (tumor shown within the oval).

FIG. 1E is a microscopy histologic image of a neoplastic mouse bladder (tumor shown within the oval).

FIG. 1F is a schematic representation of the experimental setup for the syngeneic-orthotopic mouse model for bladder cancer via intravesical (IB) instillations.

FIG. 1G is a graph showing a survival curve of mice that received different number of MB49 cells, used to determine the experimental cancer dose.

FIG. 1H is a graph showing a survival curve of mice that received single or multiple BCG IB instillations after tumor instillation (8×10⁵ MB49 cells).

FIG. 1I is a graph showing a survival curve of BCG IV vaccinated mice before tumor implantation (8×10⁵ MB49 cells). (n=18-20), two independent experiments pooled.

FIG. 1J is a histologic microscopy image showing bladders from tumor-bearing mice after PBS IB treatment at day 21 (tumor shown within the oval).

FIG. 1K is a histologic microscopy image showing bladders from tumor-bearing mice after BCG IB treatment at day 21 (tumor shown within the oval).

FIG. 1L is a graph showing a tumor weight comparison after BCG IB treatment at day 21, (n=8), two independent experiments pooled.

FIG. 2A is an in vivo pelvic magnetic resonance image (MRI) of a female C57BL/6J naïve mouse.

FIG. 2B is an in vivo pelvic MRI of tumor-bearing mice after PBS IB treatment at day 7 (arrow indicating the tumour).

FIG. 2C is an in vivo pelvic MRI of tumor-bearing mice after BCG IB treatment at day 7 (arrow indicating the tumour).

FIG. 2D is an in vivo pelvic MRI of tumor-bearing mice after PBS IB treatment at day 21 (arrow indicating the tumour).

FIG. 2E is an in vivo pelvic MRI of tumor-bearing mice after BCG IB treatment at day 21 (arrow indicating the tumour).

FIG. 2F is a graph showing a radiological axial tumoral area quantification of tumor-bearing mice after PBS or BCG IB treatment at day 21.

FIG. 3A is a graph showing BCG CFUs in urine and bladder over time post BCG instillation.

FIG. 3B is a graph showing BCG CFUs in the bladder, spleen, bone marrow (BM), Delphian lymph node (DLN) over time post BCG instillation.

FIG. 4A is a fluorescence-activated cell sorting (FACS) blot of BM-LKS+ cells in phosphate buffered saline (PBS) IB treated tumor-bearing animals at day 8.

FIG. 4B is a FACS blot of BM-LKS+ cells in PBS IB treated tumor-bearing animals at day 15.

FIG. 4C is a FACS blot of BM-LKS+ cells in PBS IB treated tumor-bearing animals at day 21.

FIG. 4D is a FACS blot of BM-LKS+ cells in BCG IB treated tumor-bearing animals at day 8.

FIG. 4E is a FACS blot of BM-LKS+ cells in BCG IB treated tumor-bearing animals at day 15.

FIG. 4F is a FACS blot of BM-LKS+ cells in BCG IB treated tumor-bearing animals at day 21.

FIG. 4G is a graph showing the kinetics of LKS+ cells in the bone marrow after BCG IB treatment of tumor-bearing mice (percentage of cells).

FIG. 5A is a graph showing the kinetics of LKS+ cells in the bone marrow after PBS or BCG IB treatment of tumor-bearing mice (absolute number of cells).

FIG. 5B is a graph showing the kinetics of the LKS+ subpopulation long-term hematopoietic stem cells HSCs (LT-HSCs) LKS⁺CD150⁺CD48⁻ in the bone marrow after PBS (square) or BCG (circle) IB treatment of tumor-bearing mice (percentage of cells).

FIG. 5C is a graph showing the kinetics of the LKS+ subpopulation short-term hematopoietic stem cells HSCs (ST-HSCs) LKS⁺CD150⁺CD48⁻ in the bone marrow after PBS (square) or BCG (circle) IB treatment of tumor-bearing mice (percentage of cells).

FIG. 5D is a graph showing the kinetics of the multipotent progenitors (MPPs; LKS⁺CD150⁻CD48⁺) in the bone marrow after PBS (square) or BCG (circle) IB treatment of tumor-bearing mice (percentage of cells).

FIG. 5E is a graph showing the kinetics of the LKS+ subpopulation LT-HSCs LKS⁺CD150⁺CD48⁻ after PBS (square) or BCG (circle) IB treatment of tumor-bearing mice (absolute number of cells).

FIG. 5F is a graph showing the kinetics of the LKS+ subpopulation ST-HSCs LKS⁺CD150⁺CD48⁻ after PBS (square) or BCG (circle) IB treatment of tumor-bearing mice (absolute number of cells).

FIG. 5G is a graph showing the kinetics of the LKS+ subpopulation MPPs LKS⁺CD150-CD48⁺ after PBS (square) or BCG (circle) IB treatment of tumor-bearing mice (absolute number of cells).

FIG. 6A is a graph showing the kinetics of neutrophils in the BM of tumor-bearing mice after PBS or BCG IB treatment (percentages).

FIG. 6B is a graph showing the kinetics of macrophages in the BM of tumor-bearing mice after PBS or BCG IB treatment (percentages).

FIG. 6C is a graph showing the kinetics of neutrophils (absolute number of cells) in the BM of tumor-bearing mice after PBS (square) or BCG (circle) IB treatment.

FIG. 6D is a graph showing the kinetics of macrophages (absolute number of cells) in the BM of tumor-bearing mice after PBS (square) or BCG (circle) IB treatment.

FIG. 6E is a graph showing the kinetics of dendritic cells (absolute number of cells) in the BM of tumor-bearing mice after PBS (square) or BCG (circle) IB treatment.

FIG. 6F is a graph showing the kinetics of dendritic cells (percentage of cells) in the BM of tumor-bearing mice after PBS (square) or BCG (circle) IB treatment.

FIG. 6G is a graph showing the kinetics of monocytes (percentage of cells) in the BM of tumor-bearing mice after PBS (square) or BCG (circle) IB treatment.

FIG. 6H is a graph showing the kinetics of monocytes (absolute number of cells) in the BM of tumor-bearing mice after PBS (square) or BCG (circle) IB treatment.

FIG. 7A is a graph showing the kinetics of neutrophils in the bladder of tumor-bearing mice after PBS or BCG IB treatment (absolute numbers).

FIG. 7B is a graph showing the kinetics of macrophages in the bladder of tumor-bearing mice after PBS or BCG IB treatment (absolute numbers).

FIG. 8A is a graph showing the kinetics of neutrophils (percentage of cells) in the bladder of tumor-bearing mice after PBS (square) or BCG (circle) IB treatment.

FIG. 8B is a graph showing the kinetics of macrophages (percentage of cells) in the bladder of tumor-bearing mice after PBS (square) or BCG (circle) IB treatment.

FIG. 8C is a graph showing the kinetics of dendritic cells (percentage of cells) in the bladder of tumor-bearing mice after PBS (square) or BCG (circle) IB treatment.

FIG. 8D is a graph showing the kinetics of dendritic cells (absolute numbers) in the bladder of tumor-bearing mice after PBS (square) or BCG (circle) IB treatment.

FIG. 8E is a graph showing the kinetics of CD4+ T cells (percentage of cells) in the bladder of tumor-bearing mice after PBS (square) or BCG (circle) IB treatment.

FIG. 8F is a graph showing the kinetics of CD8+ T cells (percentage of cells) in the bladder of tumor-bearing mice after PBS (square) or BCG (circle) IB treatment.

FIG. 8G is a graph showing the kinetics of natural killer (NK) cells (percentage of cells) in the bladder of tumor-bearing mice after PBS (square) or BCG (circle) IB treatment.

FIG. 8H is a graph showing the kinetics of monocytes (percentage of cells) in the bladder of tumor-bearing mice after PBS (square) or BCG (circle) IB treatment.

FIG. 8I is a graph showing the kinetics of CD4+ T cells (absolute numbers) in the bladder of tumor-bearing mice after PBS (square) or BCG (circle) IB treatment.

FIG. 8J is a graph showing the kinetics of CD8+ T cells (absolute numbers) in the bladder of tumor-bearing mice after PBS (square) or BCG (circle) IB treatment.

FIG. 8K is a graph showing the kinetics of NK cells (absolute numbers) in the bladder of tumor-bearing mice after PBS (square) or BCG (circle) IB treatment.

FIG. 8L is a graph showing the kinetics of monocytes (absolute numbers) in the bladder of tumor-bearing mice after PBS (square) or BCG (circle) IB treatment.

FIG. 9A is a schematic representation of the experimental design of the murine orthotopic bladder cancer model (MB49 tumor cell line) and intravesical (IB) β-Glucan instillation.

FIG. 9B is a schematic representation of the experimental design of the murine orthotopic bladder cancer model (MB49 tumor cell line) and intraperitoneal (IP) β-Glucan pre-treatment.

FIG. 9C is a graph showing a survival curve of tumor-bearing mice and PBS or β-Glucan IP pre-treatment (n=18-20), two independent experiments pooled.

FIG. 9D is a macroscopic image showing bladders of tumor-bearing mice after PBS IB treatment at day 21.

FIG. 9E is a macroscopic image showing bladders of tumor-bearing mice after β-Glucan IB treatment at day 21.

FIG. 9F is a pelvic MRI of tumor-bearing mice after PBS IB treatment at 21 days, the arrow show indicate the tumor.

FIG. 9G is a pelvic MRI of tumor-bearing mice after β-glucan IB treatment at 21 days, the arrow show indicate the tumor.

FIG. 9H is a macroscopic image of a bladder showing the neoplastic tissue (arrows).

FIG. 9I is a macroscopic image of a different side of the bladder shown in FIG. 9H.

FIG. 9J is a graph showing the tumor weight comparison after β-Glucan IB treatment schemes at 21 days, (n=8), two independent experiments pooled.

FIG. 9K is a graph showing the radiological axial tumoral area quantification of tumor-bearing mice after PBS or β-Glucan IB treatment at day 21.

FIG. 9L is a graph showing the survival curve of tumor-bearing mice after PBS or β-Glucan IB treatment (n=18-20), two independent experiments pooled.

FIG. 10A is a graph showing the kinetics of neutrophils in the BM after β-Glucan IP treatment of tumor-bearing mice (percentage of cells).

FIG. 10B is a graph showing the kinetics of neutrophils in the BM after β-Glucan IP treatment of tumor-bearing mice (absolute number of cells).

FIG. 11A is a FACS blot of neutrophils in the bladder of PBS IB treated tumor-bearing animals at day 7.

FIG. 11B is a FACS blot of neutrophils in the bladder of PBS IB treated tumor-bearing animals at day 14.

FIG. 11C is a FACS blot of neutrophils in the bladder of PBS IB treated tumor-bearing animals at day 21.

FIG. 11D is a FACS blot of neutrophils in the bladder of β-Glucan IB treated tumor-bearing animals at day 7.

FIG. 11E is a FACS blot of neutrophils in the bladder of β-Glucan IB treated tumor-bearing animals at day 14.

FIG. 11F is a FACS blot of neutrophils in the bladder of β-Glucan IB treated tumor-bearing animals at day 21.

FIG. 12A is a graph showing the kinetics of neutrophils in the bladder after β-Glucan IB treatment of tumor-bearing mice (percentage of cells).

FIG. 12B is a graph showing the kinetics of neutrophils in the bladder after β-Glucan IB treatment of tumor-bearing mice (absolute number of cells).

FIG. 13A is a graph showing the kinetics of macrophages in the bladder of tumor-bearing mice after PBS or β-Glucan IB treatment (percentages of cells).

FIG. 13B is a graph showing the kinetics of macrophages in the bladder of tumor-bearing mice after PBS or β-Glucan IB treatment (absolute number of cells).

FIG. 13C is a graph showing the kinetics of CD4+ T cells in the bladder of tumor-bearing mice after PBS or β-Glucan IB treatment (percentages of cells).

FIG. 13D is a graph showing the kinetics of CD4+ T cells in the bladder of tumor-bearing mice after PBS or β-Glucan IB treatment (absolute number of cells).

FIG. 13E is a graph showing the kinetics of CD8+ T cells in the bladder of tumor-bearing mice after PBS or β-Glucan IB treatment (percentages of cells).

FIG. 13F is a graph showing the kinetics of CD8+ T cells in the bladder of tumor-bearing mice after PBS or β-Glucan IB treatment (absolute number of cells).

FIG. 14A is a schematic representation of the experimental design of the murine orthotopic bladder cancer model (MB49 tumor cell line) and intravesical (IB) BCG/β-Glucan instillations.

FIG. 14B is a schematic representation of the experimental design of the murine orthotopic bladder cancer model (MB49 tumor cell line) and BCG IB/β-Glucan IP treatment.

FIG. 14C is a graph of the survival curve of tumor-bearing mice BCG IB/β-Glucan IP treatment (n=18-20), two independent experiments pooled.

FIG. 14D is an image of bladders of tumor-bearing mice after PBS IB treatment at day 21.

FIG. 14E is an image of bladders of tumor-bearing mice after β-Glucan/BCG IB treatment at day 21.

FIG. 14F is a pelvic MRI of tumor-bearing mice after PBS IB treatment at 21 days.

FIG. 14G is a pelvic MRI of tumor-bearing mice after β-Glucan/BCG IB treatment at 21 days.

FIG. 14H is a graph showing a tumor weight comparison after PBS or β-Glucan & BCG IB treatment at 21 days, (n=8) mice per group, two independent experiments pooled.

FIG. 14I is a graph showing the radiological axial tumoral area quantification of tumor-bearing mice after PBS or β-Glucan/BCG IB treatment at day 21.

FIG. 14J is a graph showing the survival curve after PBS or β-Glucan/BCG IB treatment (n=18-20), two independent experiments pooled.

FIG. 14K is a macroscopic image of bladders from β-Glucan/BCG IB treated animals at 80 days post tumor instillation.

FIG. 14L is a macroscopic histology image of tumor-bearing bladder after β-Glucan/BCG IB treatment at day 30.

FIG. 14M is a macroscopic histology image of tumor-bearing bladder after PBS IB at survival endpoint.

FIG. 15A is a FACS blot of BM-LKS+ cells in PBS IB treated tumor-bearing animals at day 7.

FIG. 15B is a FACS blot of BM-LKS+ cells in PBS IB treated tumor-bearing animals at day 14.

FIG. 15C is a FACS blot of BM-LKS+ cells in PBS IB treated tumor-bearing animals at day 21.

FIG. 15D is a FACS blot of BM-LKS+ cells in β-Glucan/BCG IB treated tumor-bearing animals at day 7.

FIG. 15E is a FACS blot of BM-LKS+ cells in β-Glucan/BCG IB treated tumor-bearing animals at day 14.

FIG. 15F is a FACS blot of BM-LKS+ cells in β-Glucan/BCG IB treated tumor-bearing animals at day 21.

FIG. 16A is a graph showing the kinetics of LKS+ cells in the bone marrow after PBS or -Glucan/BCG IB treatment of tumor-bearing mice (percentage of cells).

FIG. 16B is a graph showing the kinetics of LKS+ cells in the bone marrow after PBS or β-Glucan/BCG IB treatment of tumor-bearing mice (absolute number of cells).

FIG. 17A is a graph showing the kinetics of neutrophils in the BM after β-Glucan/BCG IB treatment of tumor-bearing mice (percentage of cells).

FIG. 17B is a graph showing the kinetics of neutrophils in the BM after β-Glucan/BCG IB treatment of tumor-bearing mice (absolute number of cells).

FIG. 18A is a graph showing the kinetics of neutrophils in the bladder after β-Glucan/BCG IB treatment of tumor-bearing mice (percentage of cells).

FIG. 18B is a graph showing the kinetics of neutrophils in the bladder after β-Glucan/BCG IB treatment of tumor-bearing mice (absolute number of cells).

FIG. 19A is a graph showing the kinetics of CD11b+Ly6G+dcTRAIL-R1−CD101−T1 neutrophils in the bladder after β-Glucan/BCG IB treatment of tumor-bearing mice (percentage of cells).

FIG. 19B is a graph showing the kinetics of CD11b+Ly6G+dcTRAIL-R1−CD101−T1 neutrophils in the bladder after β-Glucan/BCG IB treatment of tumor-bearing mice (absolute number of cells).

FIG. 20A is a graph showing the kinetics of CD11b+Ly6G+dcTRAIL-R1−CD101−T2 neutrophils in the bladder after β-Glucan/BCG IB treatment of tumor-bearing mice (percentage of cells).

FIG. 20B is a graph showing the kinetics of CD11b+Ly6G+dcTRAIL-R1−CD101−T2 neutrophils in the bladder after β-Glucan/BCG IB treatment of tumor-bearing mice (absolute number of cells).

FIG. 21A is a FACS blot of CD11b+Ly6G+CD101+/−dcTRAIL-R1+T3 neutrophils in the bladder of PBS treated tumor-bearing mice at day 7.

FIG. 21B is a FACS blot of CD11 b+Ly6G+CD101+/−dcTRAIL-R1+T3 neutrophils in the bladder of PBS treated tumor-bearing mice at day 14.

FIG. 21C is a FACS blot of CD11b+Ly6G+CD101+/−dcTRAIL-R1+T3 neutrophils in the bladder of PBS treated tumor-bearing mice at day 21.

FIG. 21D is a FACS blot of CD11 b+Ly6G+CD101+/−dcTRAIL-R1+T3 neutrophils in the bladder of β-Glucan/BCG IB treated tumor-bearing mice at day 7.

FIG. 21E is a FACS blot of CD11b+Ly6G+CD101+/−dcTRAIL-R1+T3 neutrophils in the bladder of β-Glucan/BCG IB treated tumor-bearing mice at day 14.

FIG. 21F is a FACS blot of CD11 b+Ly6G+CD101+/−dcTRAIL-R1+T3 neutrophils in the bladder of β-Glucan/BCG IB treated tumor-bearing mice at day 21.

FIG. 22A is a graph showing the kinetics of CD11b+Ly6G+CD101+/−dcTRAIL-R1+T3 neutrophils in the Bladder after β-Glucan/BCG IB treatment of tumor-bearing mice (percentage of cells).

FIG. 22B is a graph showing the kinetics of CD11b+Ly6G+CD101+/−dcTRAIL-R1+T3 neutrophils in the Bladder after β-Glucan/BCG IB treatment of tumor-bearing mice (absolute number of cells).

FIG. 23A is a graph showing the kinetics of monocytes in the BM after β-Glucan/BCG IB treatment of tumor-bearing mice (percentage of cells).

FIG. 23B is a graph showing the kinetics of monocytes in the BM after β-Glucan/BCG IB treatment of tumor-bearing mice (absolute number of cells).

FIG. 24A is a graph that shows the MB49 viability assessed by flow cytometry after 24 hours of coculture with PBS or β-Glucan/BCG-trained neutrophils purified from the bone marrow 14 days after IB treatment (1:1 ratio). N-Acetylcysteine (NAC) was used to scavenge ROS (5 mM) (n=4).

FIG. 24B is a microphotograph of the MB49 cells 24 hours after culture.

FIG. 24C is a microphotograph of the MB49 cells 24 hours after coculture with PBS neutrophils.

FIG. 24D is a microphotograph of the MB49 cells 24 hours after coculture with trained neutrophils.

FIG. 24E is a microphotograph of the MB49 cells 24 hours after coculture with neutrophils in the presence of NAC.

FIG. 24F is a microphotograph of the MB49 cells 24 hours after coculture with PBS neutrophils in the presence of NAC.

FIG. 24G is a microphotograph of the MB49 cells 24 hours after coculture with trained neutrophils in the presence of NAC.

FIG. 25A is a graph showing the quantification of intratumoral area of neutrophil infiltration (Ly6G+) in tumors at 7 days after MB49-GFP instillation and PBS- or β-Glucan/BCG-IB treatment (n=3).

FIG. 25B is a graph showing the Mean Fluorescent Intensity quantification of dcTRAIL-R1 in Ly6G+ areas at 7 days after MB49-GFP instillation and PBS or β-Glucan/BCG IB treatment (n=3).

FIG. 25C is a schematic representation of the experimental set up for the Intravital Microscopy (IVM) of tumoral bladder.

FIG. 25D is a screenshot from IVM recording showing the bladder tumoral border of PBS-IB treated mice at day 7 (n=3).

FIG. 25E is a screenshot from IVM recording showing the bladder tumoral border of BCG/β-Glucan-IB treated mice at day 7 (n=3).

FIG. 25F is a photograph of the bladder treated with PBS IB.

FIG. 25G is a 3D reconstruction from IVM acquisition of tumoral vascularization in the bladder of PBS IB (FIG. 25F) treated mice at day 14.

FIG. 25H is a photograph of the bladder treated with BCG/β-Glucan-IB.

FIG. 25I is a 3D reconstruction from IVM acquisition of tumoral vascularization in the bladder of BCG/β-Glucan-IB (FIG. 25H) treated mice at day 14.

FIG. 26A is a schematic representation of the experimental design for the adoptive intravesical transfer of trained neutrophils into tumor bearing mice.

FIG. 26B is a graph that shows a survival curve after PBS- or β-Glucan/BCG-trained neutrophils adoptive intravesical transfer (n=10).

FIG. 26C is a fluorescent microscopy image of whole-mount sections of bladders at 7 days after MB49 instillation and PBS-treated neutrophils (stained for CD31+).

FIG. 26D is a close up of FIG. 26C.

FIG. 26E is a fluorescent microscopy image of whole-mount sections of bladders at 7 days after MB49 instillation and BCG/β-Glucan-trained neutrophils (stained for CD31+).

FIG. 26F is a close up of FIG. 26E.

FIG. 26G is a graph showing the quantification of blood vessels area (CD31+) in tumors at 7 days after MB49 instillation and PBS- or β-Glucan/BCG-trained neutrophils (n=5).

FIG. 26H is a schematic representation of the experimental design for the depletion of neutrophils in tumor bearing mice.

FIG. 26I is a graph showing the survival curve after neutrophils depletion and PBS- or β-Glucan/BCG-IB treatment in tumor bearing mice (n=8-10).

FIG. 26J is a FACS blot showing the isotype control with blood of tumor-bearing animals at 21 days.

FIG. 26K is a FACS blot showing neutrophils depletion in the blood of PBS/BCG/β-Glucan IB treated tumor-bearing animals at 21 days (anti-Ly6G).

FIG. 26L is a FACS blot showing the isotype control with the bladder of tumor-bearing animals at 21 days.

FIG. 26M is a FACS blot showing neutrophils depletion in the bladder of PBS/BCG/β-Glucan IB treated tumor-bearing animals at 21 days (anti-Ly6G).

FIG. 27A is a schematic representation of BCG and/or β-Glucan systemic treatment and MB49 subcutaneous tumor model.

FIG. 27B is a photograph that shows the macroscopic appearance of subcutaneous MB49 tumors across all experimental conditions at 20 days after MB49 injection (n=4).

FIG. 27C is a graph that shows the tumor weight comparison across all experimental conditions at 20 days after MB49 injection (n=4).

FIG. 27D is a schematic representation for the MB49 subcutaneous tumor model and BCG and/or β-Glucan intratumoral therapy.

FIG. 27E is a graph showing the tumor weight comparison across all experimental conditions at 7 days after MB49 injection (n=4).

FIG. 27F is a graph showing the percentage of tumor infiltrating neutrophils across all experimental conditions (n=4).

FIG. 27G is a graph showing the percentage of protumoral neutrophils (T3) across all experimental conditions (n=4).

FIG. 27H is a schematic representation for the orthotopic melanoma model (B16-F10) and BCG and/or β-Glucan intratumoral therapy.

FIG. 27I is a photograph showing the macroscopic appearance of subcutaneous MB49 tumors across all experimental conditions at 14 days after B16-F10 injection (n=5).

FIG. 27J is a graph showing the tumor weight comparison across all experimental conditions at 14 days after B16-F10 injection (n=5).

FIG. 27K is a graph showing the percentage of tumor infiltrating neutrophils in all experimental conditions (n=4).

FIG. 27L shows FACS blots of Siglec-F^{io} CD62L^{hi} neutrophils across all treatment conditions (shown as percentage of CD45+ cells, n=4).

FIG. 27M is a graph showing the percentage of tumor infiltrating Siglec-F^{io} CD62L^{hi} neutrophils in all experimental conditions (n=4).

FIG. 28A is a graph showing the percentages of neutrophils and Sca-1^{hi} neutrophils in blood after MB49 subcutaneous injection across all experimental conditions (shown as percentage of viable cells n=4).

FIG. 28B is a graph showing the percentages of neutrophils and Sca-1^{hi} neutrophils in BM after MB49 subcutaneous injection across all experimental conditions (shown as percentage of viable cells n=4).

FIG. 28C is a graph showing the percentages of neutrophils and Sca-1^{hi} neutrophils in tumor after MB49 subcutaneous injection across all experimental conditions (shown as percentage of viable cells n=4).

FIG. 29A is a FACS blot of Sca-1^{hi} blood neutrophils treated with PBS at day 7 (shown as percentage of neutrophils, n=4).

FIG. 29B is a FACS blot of Sca-1^{hi} blood neutrophils with β-glucan treatment conditions at day 7 (shown as percentage of neutrophils, n=4).

FIG. 29C is a FACS blot of Sca-1^{hi} blood neutrophils with BCG treatment conditions at day 7 (shown as percentage of neutrophils, n=4).

FIG. 29D is a FACS blot of Sca-1^{hi} blood neutrophils with β-glucan+BCG treatment conditions at day 7 (shown as percentage of neutrophils, n=4).

FIG. 30A is a graph the percentage of tumor infiltrating Sca-1^{hi} blood neutrophils in all experimental conditions (n=4).

FIG. 30B is a graph showing the percentages of LKS+ cells in the bone marrow of mice bearing melanoma across all the experimental conditions (n=4).

FIG. 30C is a graph showing the percentages of neutrophils in the bone marrow of mice bearing melanoma across all the experimental conditions (n=4).

FIG. 30D is a graph showing the percentages of T1 neutrophils (CD11 b+Ly6G+dcTRAIL-R1−CD101−) in the MB49 subcutaneous tumor after PBS/β-Glucan/BCG SC treatments (shown as percentage of viable cells and absolute number n=4).

FIG. 30E is a graph showing the percentages of T2 neutrophils (CD11b+Ly6G+dcTRAIL-R1−CD101⁺) in the MB49 subcutaneous tumor after PBS/β-Glucan/BCG SC treatments (shown as percentage of viable cells and absolute number n=4).

### DETAILED DESCRIPTION

The administration of systemic BCG (intravascular, IV) enhances the innate immune response and confers host protection against both homologous (Mycobacterium tuberculosis or M. tuberculosis) and heterologous (influenza virus) infections. Importantly, BCG protection is mediated by epigenetic reprogramming of hematopoietic stem cells (HSC) in the bone marrow (BM), which is transmitted to progenitor and innate cells thereby inducing central trained immunity. The repeated installations of BCG in both murine model and patients with bladder cancer results in long-term reprogramming of HSCs and progenitor cells and the generation of effective trained immunity against cancer. However, although BCG has shown efficacy in enhancing the anti-tumor immunity in bladder cancer, up to 50% of patients remain unresponsive to the BCG immunotherapy.

It was surprisingly found that the addition of the adjuvant β-glucan to the BCG intravesical therapy, significantly increased the survival rate in the bladder cancer animal model through the generation of a unique subset of neutrophils. Specifically, across mouse models and in multiple human cancers, both immature and mature neutrophils infiltrate the tumor and differentiate into transitional T1 and T2 populations with antitumor capacity. However, the tumor microenvironment subsequently induces a terminal differentiation into the T3 pro-tumor neutrophil, a predictor of poorer prognosis in patients with solid tumors. A synergistic effect of BCG and β-glucan is demonstrated in the Example section below, where the neutrophil population is reprogrammed to resist their conversion into pro-cancer (T3) in the tumour environment. Without being bound by theory, it is anticipated that this reprogramming will significantly decrease the likelihood of relapse following remission or successful treatment with the combination of BCG and β-glucan.

Similarly to bladder cancer, melanoma, lung adenocarcinoma, head and neck squamous cell cancer, pancreatic adenocarcinoma, low-grade gliomas, esophageal carcinoma and cervical squamous cell carcinoma are characterized by a T3 neutrophil profile in the tumor microenvironment (Ng, Melissa S F, et al. “Deterministic reprogramming of neutrophils within tumors.” Science 383.6679 (2024): eadf6493). Accordingly, the present combination of β-glucan and BCG can also be used for the treatment of cancers with a T3 neutrophil profile in the tumor microenvironment such as those mentioned above. Accordingly, the present disclosure is not limited to bladder cancer and melanoma although those were the two cancer models used for the experiments.

The term “BCG vaccine” as used herein refers to Bacillus Calmette-Guérin which is an attenuated (virulence-reduced) strain of Mycobacterium bovis, that has lost its ability to cause disease in animals and humans. There are different strains of the BCG vaccine, these are for example the TICE strain, Frappier strain, Danish strain 1331, Glaxo 1077 strain, Tokyo 172-1 strain, Pasteur 1173 P2 strain, Moscow-I strain, RIVM strain, Connaught strain, Russia strain or Moreau strain. The TICE strain is specifically Mycobacterium tuberculosis var. BCG strain Trudeau Mycobacterial Collection (TMC) 1028 (also available at the American Type Culture Collection (ATCC) number 35743) which was isolated from bovine milk. TICE is the strain of BCG used for the treatment of bladder cancer in Canada and US.

β-glucans are polysaccharides of D-glucose monomers linked through β-glycosidic bonds, and are widely present in yeast, fungi (including mushrooms), some bacteria, seaweeds, and cereals (oat and barley). Among the various structural conformations of β-glucan linkages, including β (1,3), β (1,4) and β (1,6), only molecules with a β-(1,3)-linked D-glucose backbone have immunomodulatory properties. Accordingly, the term “β-glucan” as used henceforth, and unless specified otherwise, is defined as a β-glucan that has a β (1,3) glucose backbone. Preferred β-glucan are obtained from the cell wall of yeast and fungi. The cell wall β-glucans are characterized by having a β (1,6) branching that links the β (1,3) glucose backbones (i.e. side branching). In some embodiments, the β-glucan is a yeast β-Glucan, a polymer of β-(1-3)-D-glucopyranosyl units with branching at β-(1-6)-D-glucopyranosyl. The chemical formula below shows repeating units labelled n and m of β-(1-3) glucose linked by a β-(1-6) branching linkage glucose (n and m are for example integers that can be fairly large such that the below identified molecular weights are obtained).

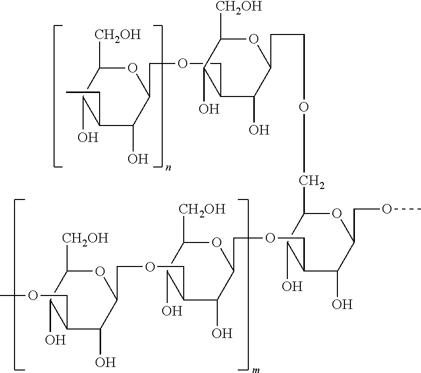

The repeating units yield a polymer generally with a size of from 100 kDa to 900 kDa. Generally, cell wall β-glucan have a molecular weight in the range of 150 kDa to 300 kDa for both yeast and fungi.

Studies in the 1960s initially proposed the antitumoral potential of β-glucan. More recently it was identified that fungal β-Glucan reprograms HSCs in the BM generating trained immunity with remarkable increased survival against pulmonary infection via monocytes/macrophages (myelopoiesis) or cancer via neutrophils (granulopoiesis) and reactive oxide species (ROS) production, which is facilitated by β-glucan recognition via the Dectin-1 receptor on neutrophils. Moreover, granulopoiesis in the BM results in generation of neutrophils at various stages of maturation (immature vs mature) with distinguished functional capacity that can be modulated by β-glucan.

As described in greater details in the example section below, the impact of intravesical treatment of bladder cancer and melanoma bearing mice with BCG, β-glucan, and its combination on HSCs in the BM and the subsequent generation of innate immune cells with antineoplastic phenotype was investigated. Surprisingly, it was found that while the treatment with BCG or β-glucan (each alone) resulted in 50% increase in survival, the combination of both BCG and β-glucan enhanced the survival to 100% with no detection of tumors in the bladder at the latest timepoints. Interestingly, BCG or β-glucan reprograms HSCs predominately towards granulopoiesis and the generation of trained neutrophils. However, the magnitude and maintenance of trained neutrophils was significantly augmented in the dual therapy with BCG/β-glucan. Thus, a synergistic effect was observed in the combination of BCG and β-glucan. Flow cytometry analysis of neutrophil in the bladder revealed that the treatment with BCG/β-glucan, induces a predominant immature phenotype, characterized by its antitumoral capacity. Following their infiltration into the bladder, these neutrophils were able to resist converting to pro-tumor neutrophils (T3). The present findings demonstrate that the combination of BCG and β-glucan therapy not only amplifies the individual benefits of each anti-cancer agent but also addresses challenges associated with non-responsiveness and adverse effects of repeated BCG installations due to the observed synergistic effect.

### Example

### Mice for the Mice Model of Bladder Cancer (BC)

C57BL/6 mice were purchased from the Jackson Laboratory. All animals were housed and inbred at the animal facility of the Research Institute of McGill University under specific-pathogen-free conditions with access to food and water (temperature of 21° C. (±1° C.), relative humidity of 40-60% (±5%) and light cycle of 12-h ON, 12-h OFF (daily cycle)). Eight- to ten-week-old females were used for all the experiments.

### Cancer Cell Lines

MB49 cells, which are a bladder cancer cell line, were purchased from the American Type Culture Collection (ATCC). Cells were cultured with complete Dulbecco's Modified Eagle Medium (DMEM), containing 10% inactivated fetal bovine serum (FBS), Glutamax™ (Sigma) and penicillin/streptomycin (Sigma) and were always used with less than 10 passages from thawing. B16-F10 cells were provided by the University of Montreal, they were cultured in complete Roswell Park Memorial Institute (RPMI), supplemented with 10% inactivated fetal bovine serum (FBS), Glutamax (Sigma) and penicillin/streptomycin (Sigma) and were always used with less than 10 passages from thawing. Cells were cultured at 37° C. in a humidified atmosphere of 5% CO₂. All the stocks used were confirmed to be negative for mycoplasma by annual testing using MycoAlert™ Plus (Lonza).

Orthotopic Instillation of MB49 Cells into the Mice to Obtain the BC Mice Model

Eight to ten-week-old female mice were anesthetized with isoflurane and intravesically instilled with 50 μL of 0.01% poly-L-lysine (Sigma) solution using a 24G catheter (BD Insyte). The poly-L-lysine solution was maintained in the bladder for 30 min, and the catheter was removed. The bladder was emptied by applying gentle pressure, then 50 μL of a solution containing 8×10⁵ MB49 cells was intravesically instilled and retained in the bladder for 1 hour, after which the bladder was emptied, and the mice were allowed to recover from anesthesia. The animals were monitored and weighed every other day and evaluated based on weight loss, presence of haematuria, palpable signs of growing tumor, and general behavior, and were euthanized when reaching a predefined endpoint criterion.

### Subcutaneous Tumor Models

Eight- to ten-week-old female and male mice were anesthetized with isoflurane and injected with 5×10⁴ MB49 cells or 3×10⁵ B16-F10 cells subcutaneously in 200 μL of PBS into the flank.

### BCG Vaccine

TICE™ BCG (American type culture collection (ATCC) Number 35743) herein referred to as BCG-TICE was grown in 7H9 broth (BD Biosciences) supplemented with 0.2% glycerol (Wisent), 0.05% Tween™ 80 (BD Biosciences) and 10% albumin-dextrose-catalase (ADC) under constant shaking at 37° C. For vaccination or intravesical instillation, bacteria in log growing phase (optical density at 600 nm (OD₆₀₀) of 0.4-0.9) were centrifuged (2,000 g, 10 min) and resuspended in sterile phosphate buffered saline (PBS). A single-cell suspension was obtained by passing the bacteria 10-15 times through a 22-gauge needle. Animals were treated with 1×10⁶ CFUs in 100 μL of sterile PBS intravenously or 5×10⁶ CFUs BCG-TICE in 50 μL of sterile PBS intravesically.

### β-Glucan

β-Glucan (from baker's yeast S. cerevisiae, Sigma-Aldrich, chemical abstract service number 9012-72-0) was suspended in sterile PBS, 20 mg per mL, a homogeneous suspension was obtained by passing the mixture 10 times through a 22G needle (Terumo), each animal received 1 mg of β-Glucan per dose.

### Intravesical Treatments

Mice were anesthetized and intravesically instilled using a 24G catheter with 50 μL of a solution containing 5×10⁶ bacteria and/or β-Glucan (1 mg per animal) diluted in phosphate buffered saline (PBS) and the catheter was kept inserted for one hour. The bladder was emptied, and the mice were allowed to recover from anesthesia under a warming lamp.

### Flow Cytometry

Bladders were digested with 100 U of collagenase D (1 mg/mL), Liberase™ (0.20 U/mL) and DNAse (1 mg/mL) (Sigma) for 1 h at 37° C. Then the digested bladders were filtered through a 70-μm nylon mesh and red blood cells were lysed with an ammonium-chloride-potassium lysing buffer. Blood was obtained through cardiac puncture, and directly stained with conjugated antibodies after which red blood cells were lysed. Bone marrow was obtained by snipping off the epiphysis of the femurs and spinning down the bones for 15 seconds in a microtube. Cells were stained with viability dye eFluor-506™ (Invitrogen; 1:1,000 dilution) for 30 min at 4° C., then stained with anti-CD16/32 (BD Biosciences; 1:200 dilution) in 0.5% bovine serum albumin (BSA)/PBS buffer to block nonspecific binding with Fc receptors for 10 min at 4° C. Cells were then stained for extracellular markers for 30 min at 4° C. Consistent identification of cells was performed using anti-Ter-119, anti-CD11b, anti-CD5, anti-CD4, anti-CD8a, anti-CD45R, and anti-Ly6G/C (clone RB6-8C5), all biotin-conjugated (BD Bioscience), Streptavidin—APC-Cy7 (eBioscience), anti-c-Kit—APC (eBioscience), anti-Sca-1—PE-Cy7 (eBioscience), anti-CD150—eFluor450 (eBioscience), anti-CD48—PerCP-eFluor710 (BD Bioscience), anti-Flt3—PE (BD Bioscience), and anti-CD34—FITC (eBioscience) (all 1:100), fluorescein isothiocyanate (FITC) conjugated anti-CD45.2 (1:200 dilution), Bright Ultra Violet™ 605 (BUV605) conjugated anti-CD11b, phycoerythrin (PE) conjugated anti-CD3 (1:200 dilution), eFluor450™ conjugated anti-CD4, Alexa Fluor™ 700 conjugated anti-CD8 (1:200 dilution) and Brilliant Violet™ 786 (BV786) conjugated anti-SiglecF Brilliant Violet™ 786 (BV785) conjugated anti-Ly6G, phycoerythrin (PE) conjugated anti-dcTRAIL-R1, PeCy7 conjugated anti-CD101 (all 1:200 dilution). Intracellular staining was performed with Cytofix/Cytoperm™ kit (BD Biosciences). Flow cytometry was performed using a BD LSR Fortessa™ X-20 (BD Biosciences) with FACSDiva version 8.0.1 (BD Biosciences). Analysis was performed using FlowJo version 10.8.1.

### CFUs

Organs were homogenized in 1 mL 7H9 broth (BD Biosciences) supplemented with 0.2% glycerol (Wisent), 0.05% Tween80™ and 10% antibody-drug conjugates (ADC) using homogenizer probes (Omni International). Serial dilutions in PBS with 0.05% Tween80™ were plated on 7H10 agar plates with 10% oleic albumin dextrose catalase (OADC) enrichment and PANTA™ (BD). Plates were then incubated at 37° C. and counted after 21 days.

### Magnetic Resonance Imaging (MRI) Scanning

Mice were anesthetized with isoflurane and introduced in a 7T Bruker™ 70/30 MRI scanner and placed in the supine position in an MRI-compatible bed. The animals were maintained at about 37° C. using an air-warming system (SA Instruments Inc.). The total time that each animal was in the magnet, under anesthesia, was approximately 1 hour. Following completion of scanning, animals were given approximately 0.5 mL of sterile, warmed saline subcutaneously, and monitored during recovery from anesthesia under a warming lamp.

### Statistical Analysis

Data are presented as the mean±standard error of the mean (s.e.m.). Statistical analyses were performed using GraphPad™ Prism v10 software (GraphPad™). Unless stated otherwise, statistical differences were determined using a two-sided log-rank test (survival studies), one-way ANOVA™ followed by Tukey's multiple-comparisons test or two-way ANOVA™ followed by Tukey's multiple-comparisons test (fluorescence-activated cell sorting (FACS) data).

### Statistics and Reproducibility

All experiments were reproduced independently two or three times to confirm the reproducibility of the present findings.

### Intravesical BCG Treatment Expands HSCs Via Access in the Bone Marrow

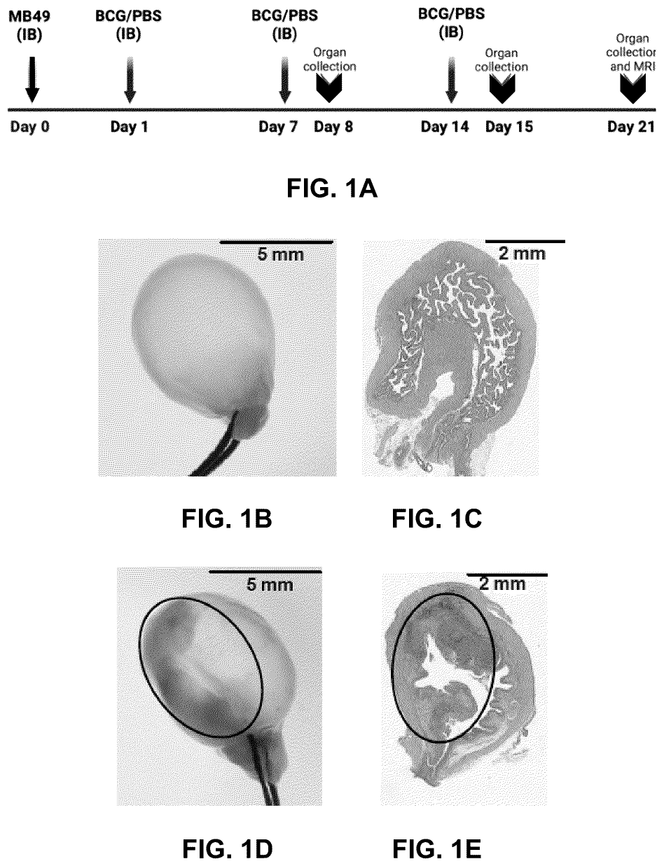

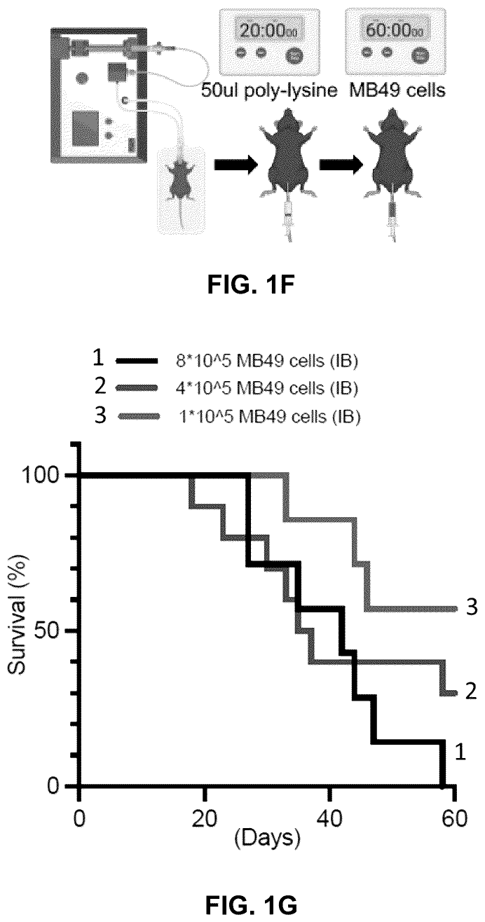

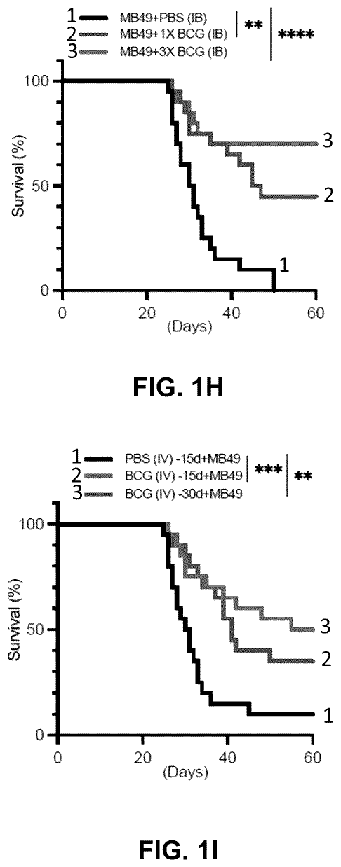

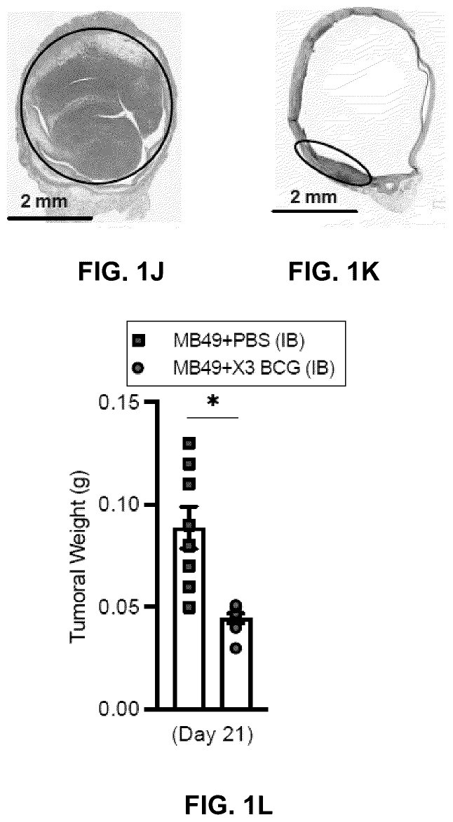

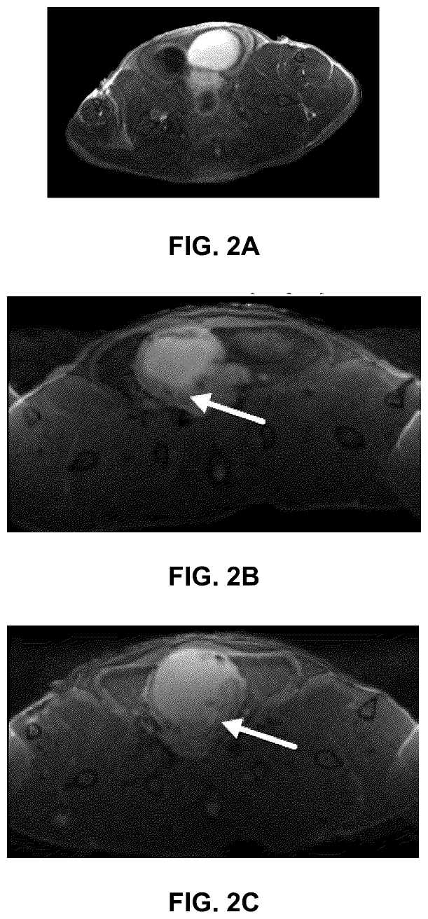

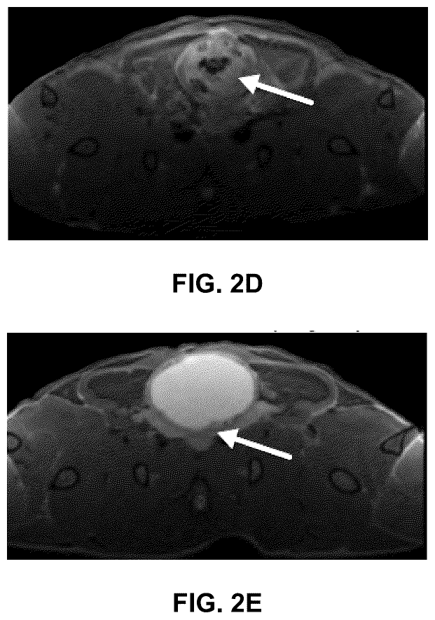

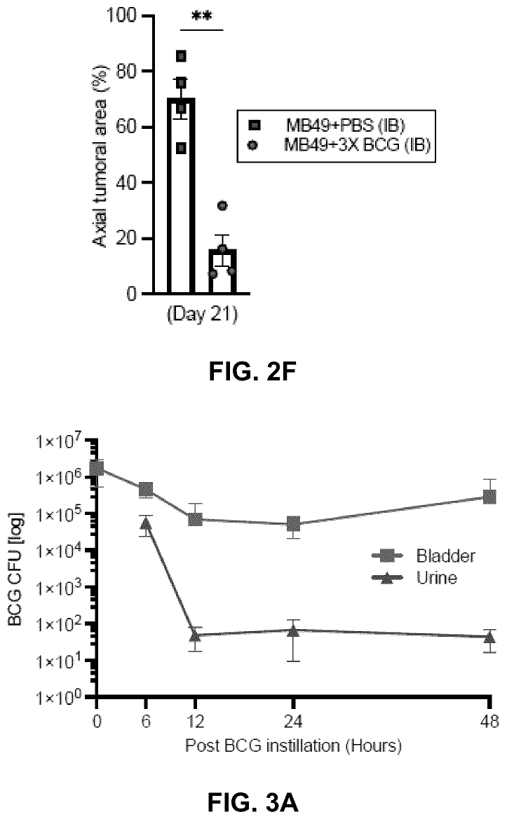

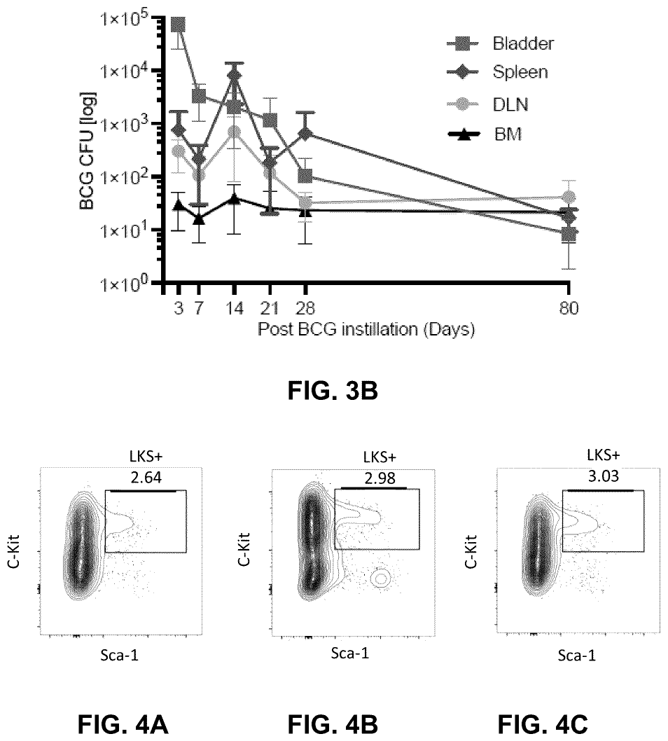

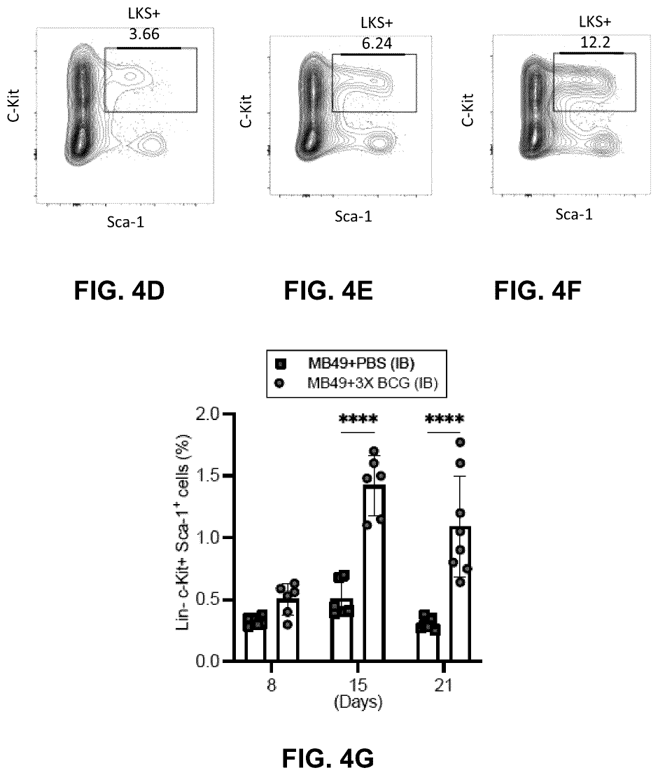

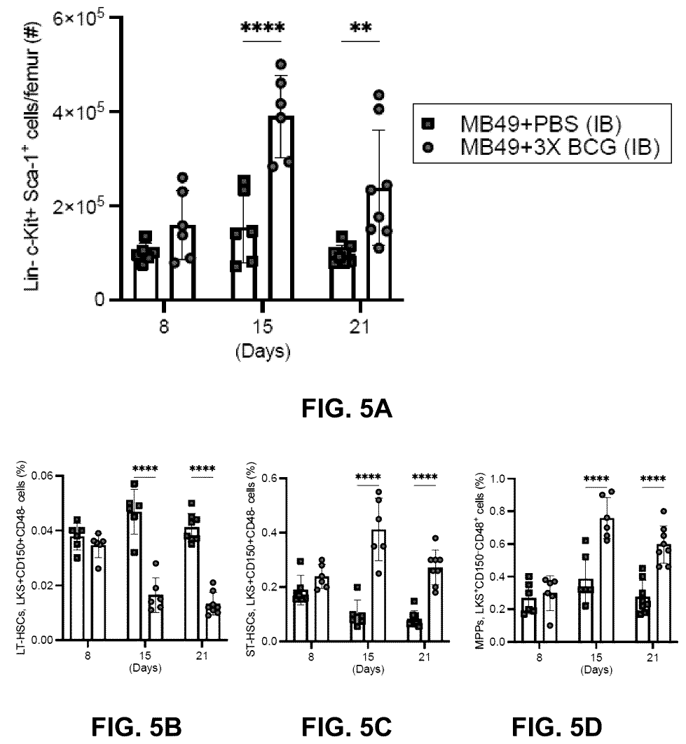

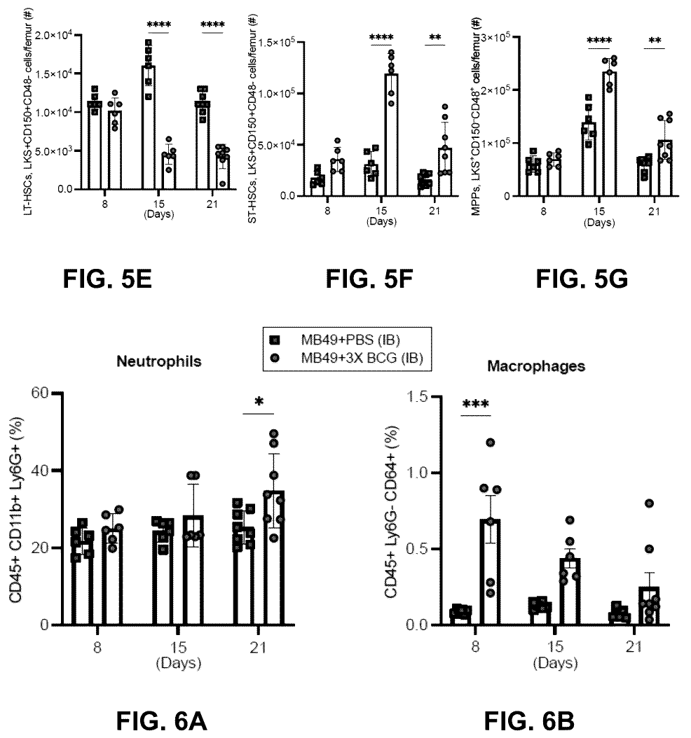

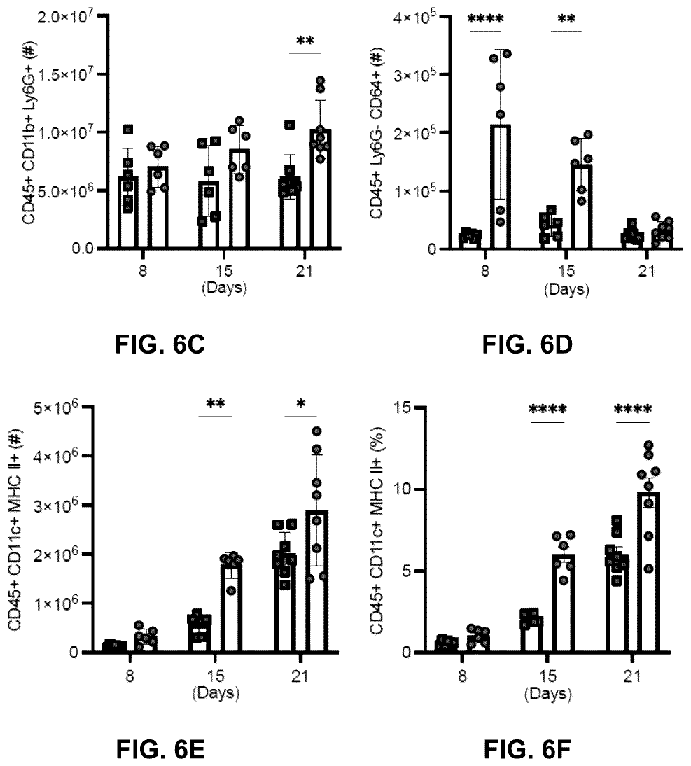

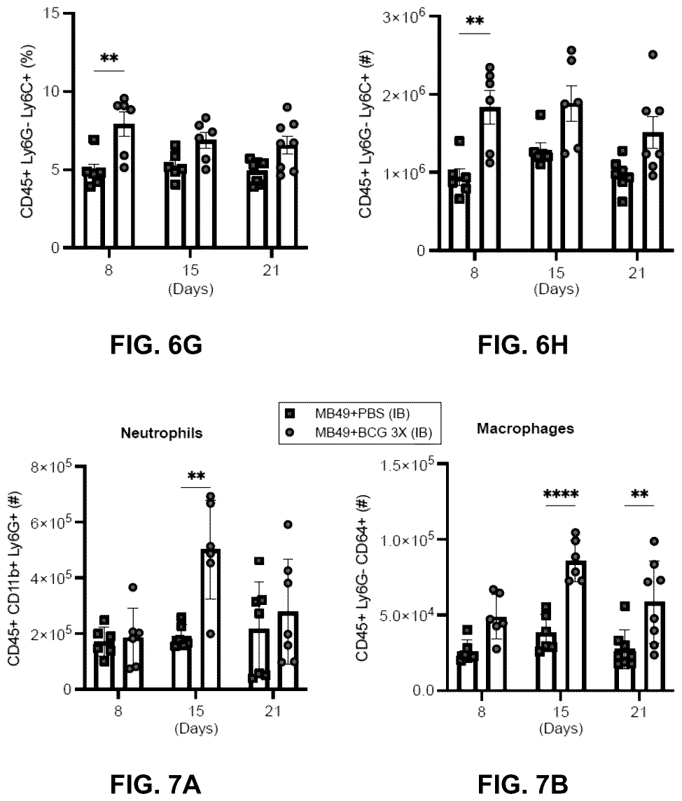

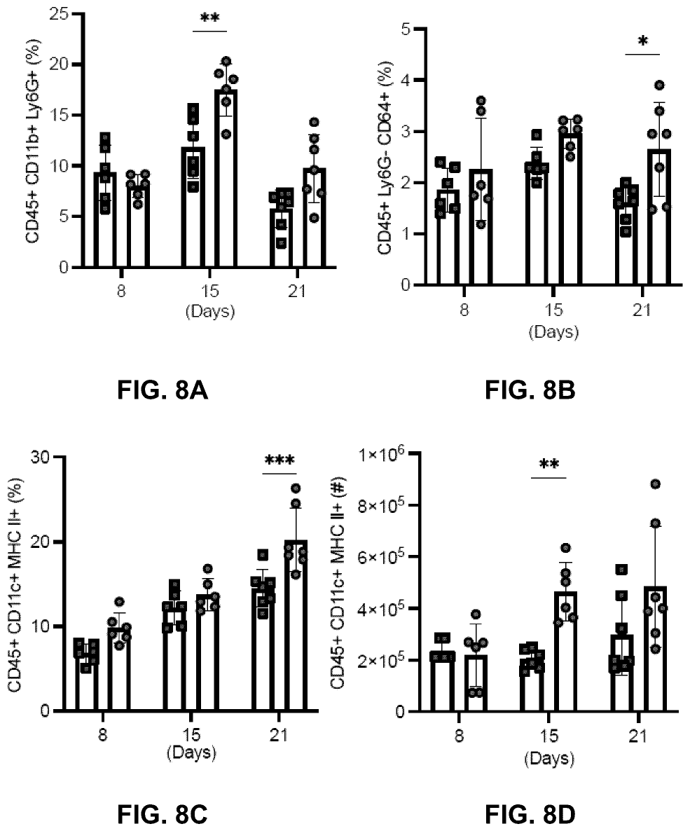

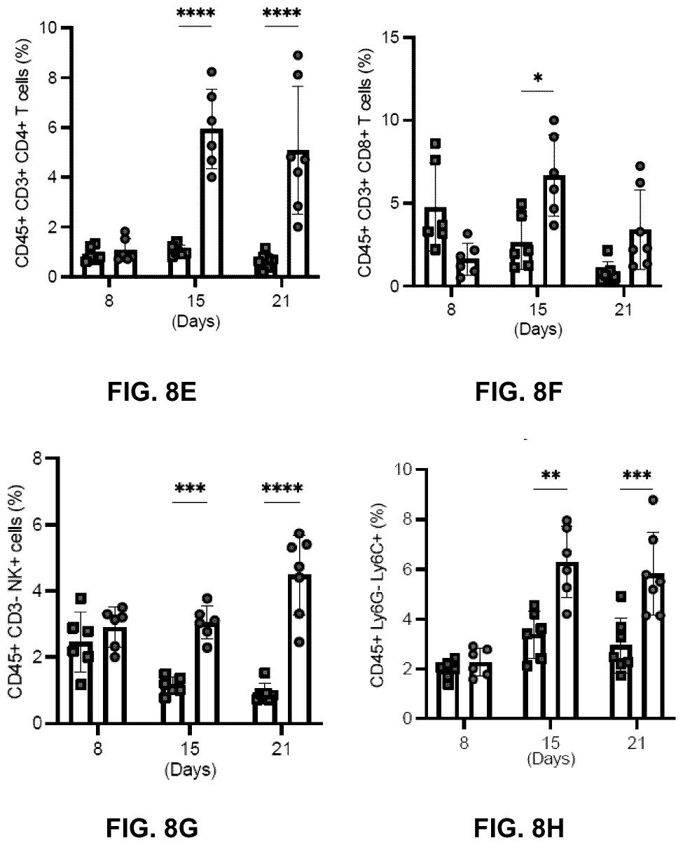

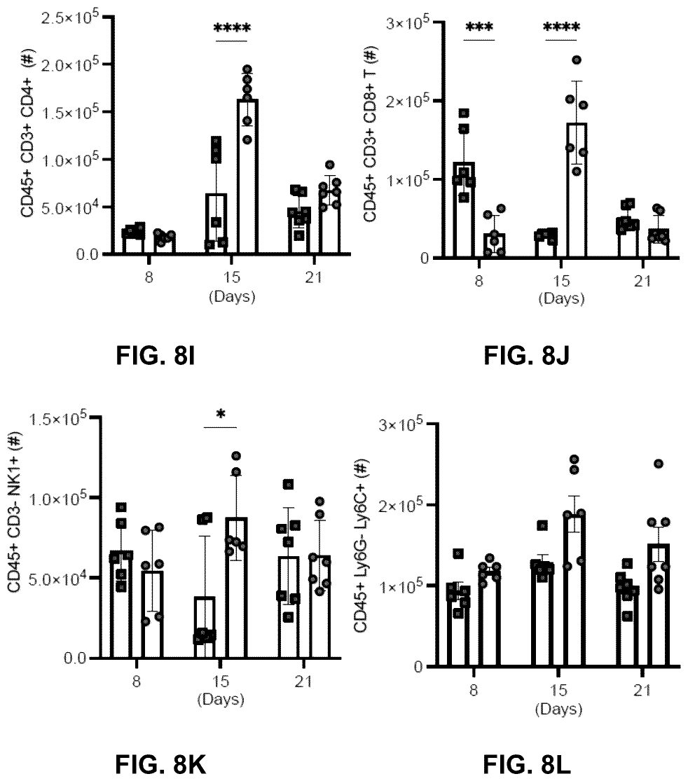

The intradermal administration of BCG in humans, as well as the intravenous vaccination (IV) in non-human primates and mice, has been shown to induce reprogramming of HSCs in the BM. Following systemic administration of BCG (IV), BCG reaches the bone marrow which results in the expansion and epigenetic reprogramming of both HSCs and progenitor cells (Lineage—Sca1+cKIT+; LKS+). This reprogramming generates trained mature myeloid cells via interferon-gamma (IFN-γ) signaling, which exhibits a distinct transcriptomic and epigenomic signature associated with sustained innate immune protection against infections. Thus, the present initial objective was to assess whether the intravesical (IB) BCG administration (the clinical TICE-strain) expands HSCs in the BM to generate central trained immunity against bladder cancer. Initially, a robust syngeneic-orthotopic mouse model was established and optimized for bladder cancer through intravesical (IB) instillations (FIGS. 1A-1G). Mice subjected to either single or three doses of BCG-IB exhibited a significant 50-70% survival rate, contrasting with no survival observed in the control animals treated with PBS-IB (FIGS. 1H-1I). This phenomenon was further characterized by a substantial reduction in tumoral volume (FIGS. 1J-1L), which was confirmed through in vivo MRI assessments (FIGS. 2A-2F) and histopathology (FIGS. 1J-1K). To test the hypothesis that this protective effect was partially mediated through direct HSC reprogramming by BCG in the BM, mice were subsequently vaccinated systemically with BCG (IV) and induced bladder cancer after 2 or 4 weeks. Interestingly, BCG-IV vaccination enhanced survival by 40-50% (FIG. 1I). This suggests that BCG should also gain access to the BM following intravesical vaccination. Assessment of BCG growth in different organs revealed that following BCG-IB, while some BCG was eliminated in the urine during the first 48 h (FIG. 3A), the bacteria reached the spleen, draining lymph nodes, and BM (FIG. 3B). The presence of BCG in the BM was associated with significant expansion of the frequency and absolute number of the LKS⁺ population at days 14 and 21 following the first BCG-IB treatment (FIGS. 4A-4G and 5A). Further characterization of HSC subpopulations in the BM showed that the expansion of the LKS⁺ population was attributed to increases in the proportions of the short-term HSCs (ST-HSCs; LKS⁺CD150⁺CD48⁺), and multipotent progenitors (MPPs; LKS⁺CD150⁻CD48⁺), but a reduction in the proportions of the long-term HSCs (LT-HSCs; LKS⁺CD150⁺CD48⁻) (FIGS. 5B-5G). This led to increased frequency and absolute numbers of neutrophils, dendritic cells, and monocytes in the BM (FIGS. 6A-6H) and bladder (FIGS. 7A-7B). The frequency and total cell numbers of other innate cells (macrophages, DCs, and NK cells) as well as lymphocytes (CD4⁺ and CD8⁺ T cells) were also increased in the bladder particularly 15 days after the tumor implantation (FIGS. 8A-8L).

Importantly, using both single and serial engraftment model, the long-term nature of the HSC imprinting induced by BCG-IV and BCG-IB treatments was demonstrated. Furthermore, in two distinct cohorts of bladder cancer patients, similar epigenetic and transcriptional reprogramming was observed, including significant upregulation of genes associated with neutrophil function, highlighting these conserved mechanisms between mice and humans. Collectively, these data indicate that following BCG-IB treatment in the murine model of bladder cancer, BCG accesses the BM and reprograms HSCs and progenitor cells towards granulopoiesis and myelopoiesis, leading to increased generation of trained monocytes, dendritic cells and neutrophils in the BM and subsequently these trained cells infiltrate in the tumor. This reprogramming indicates a broader immune response, including T cell-mediated mechanisms, contributing to an enhanced anti-tumor immunity.

### Intravesical β-Glucan Treatment Enhances Tumor Clearance and Survival

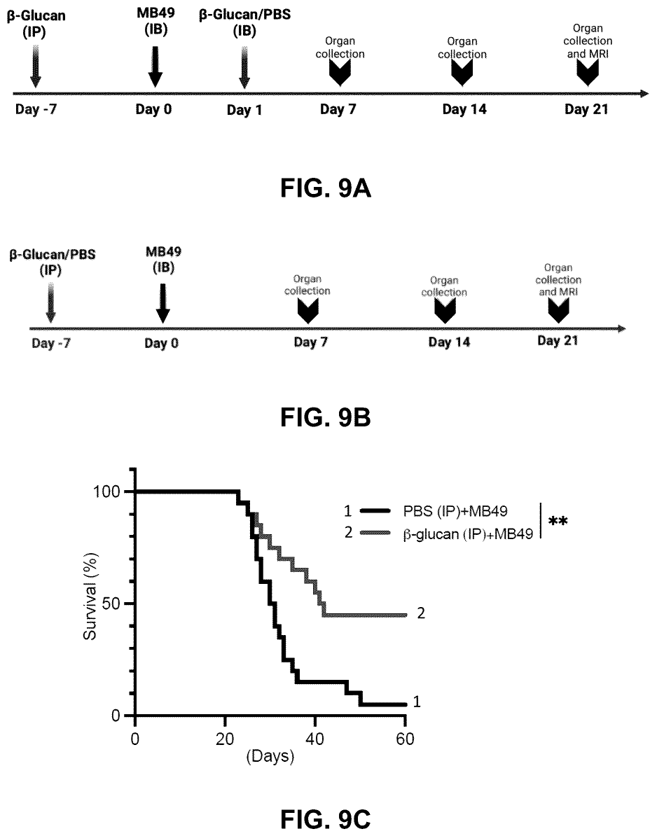

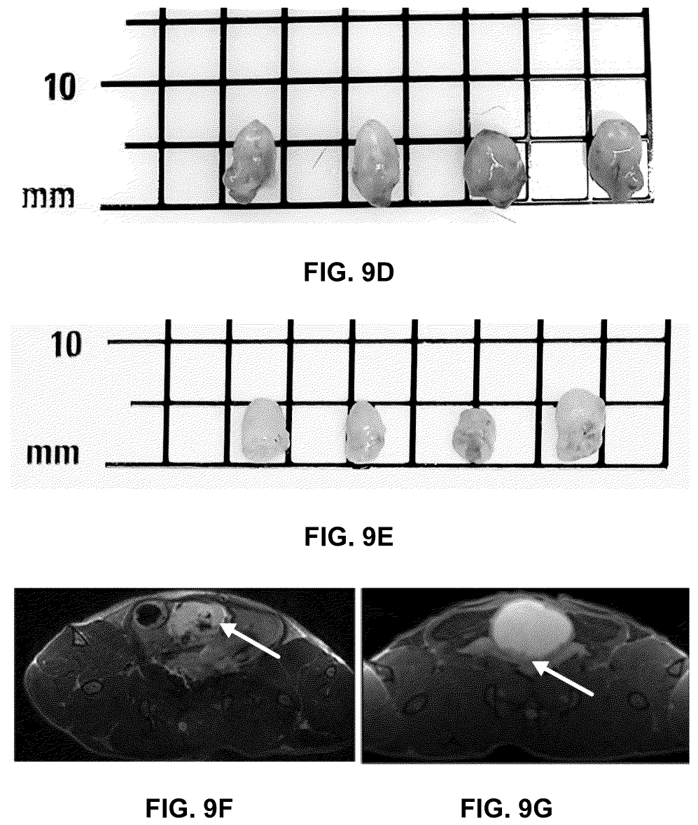

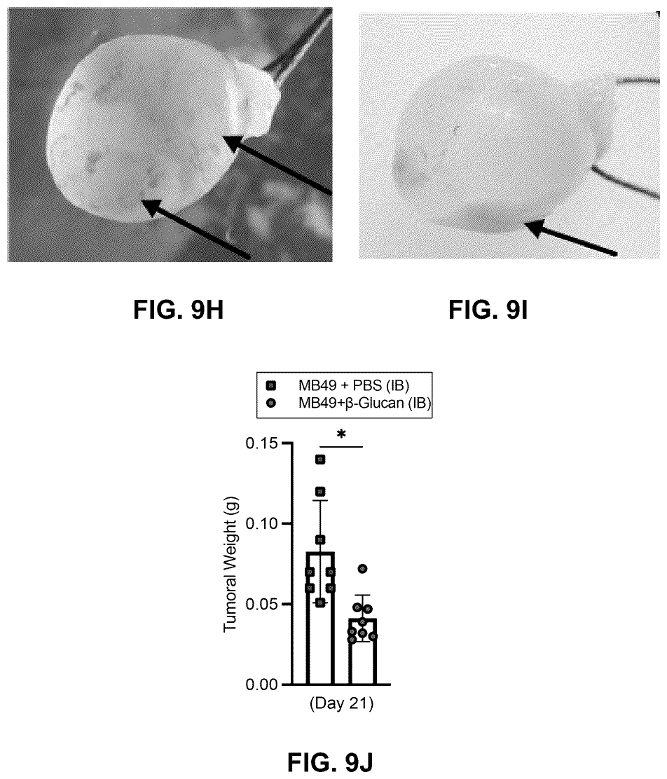

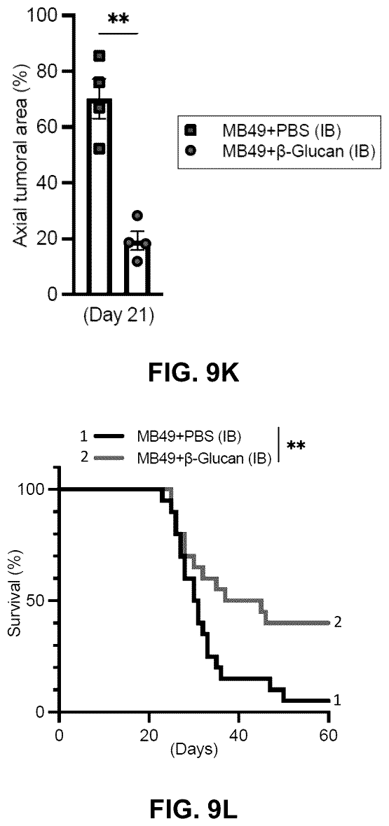

The systemic administration (intraperitoneal) of β-glucan reprograms HSCs and enhances immunity against infections or cancer. Notably, these modifications in progenitor cells result in sustained and enhanced responsiveness when subjected to subsequent inflammatory challenges. The intravesical administration of β-glucan in the mice model was investigated. It was found that β-glucan administration induces antitumoral effects in the mouse orthotopic bladder cancer model (FIG. 9A). Systemic (IP) administration of β-glucan 7 days prior to instillation of bladder cancer enhanced survival by 45% compared to the PBS-control group (FIGS. 9B-9C). More importantly, it was found that the intravesical β-glucan treatment after the tumoral implantation, was also capable of suppressing tumoral growth, as evidenced by pathology, MRI and tumoral weight (FIGS. 9D-9K). The suppression of the bladder tumor growth by β-glucan-IB was associated with increased survival by 40% (FIG. 9L).

To further validate the essential role of neutrophils in β-Glucan tumor reduction and enhanced survival, the neutrophils were depleted in tumor bearing mice treated with PBS- or β-Glucan-IB (FIG. 9I). Neutrophil depletion abolished the therapeutic effect of the β-Glucan intravesical treatment in the tumor-bearing animals, reducing survival from 40% to 10% (FIG. 9J). This indicates that intravesical β-Glucan treatment promotes granulopoiesis and the generation neutrophils that infiltrate the bladder reducing tumor growth and enhancing survival.

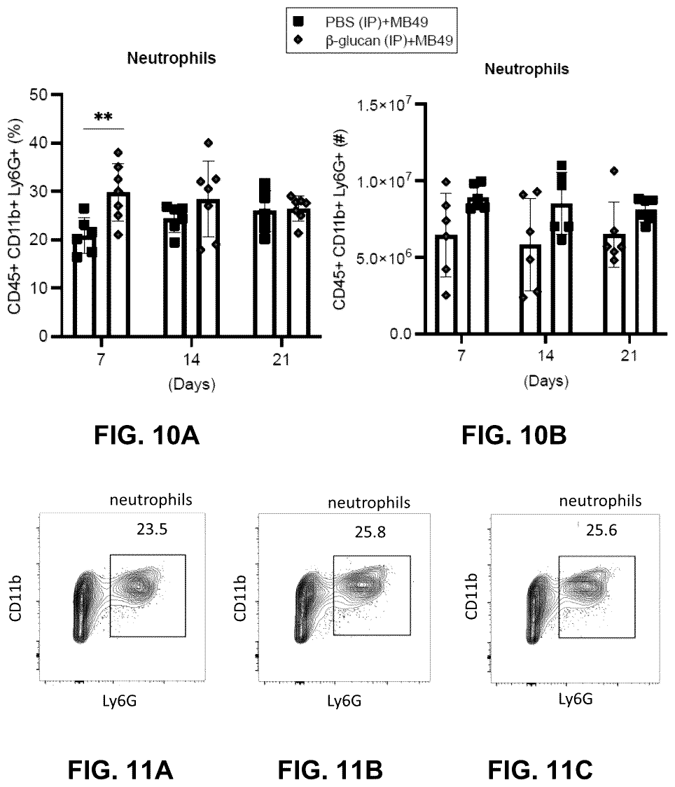

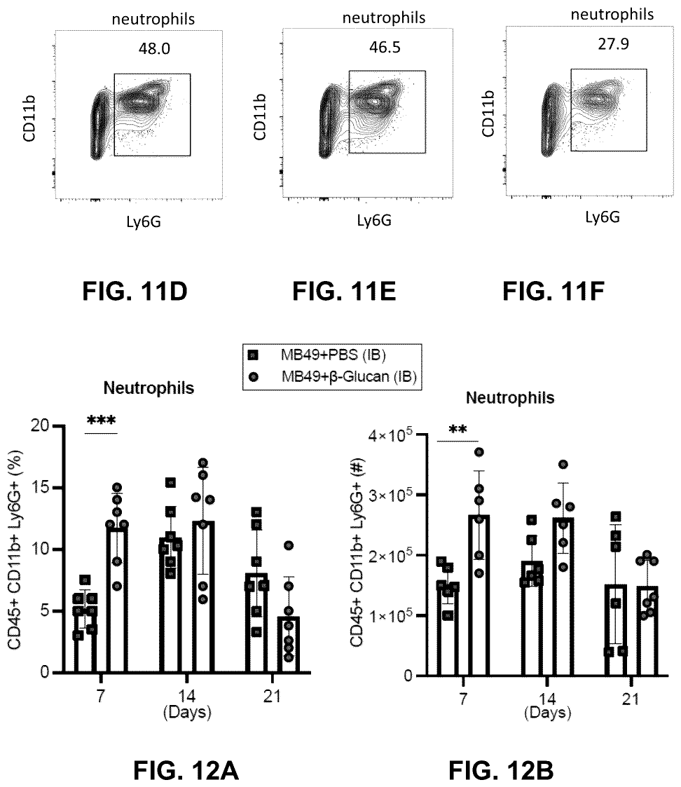

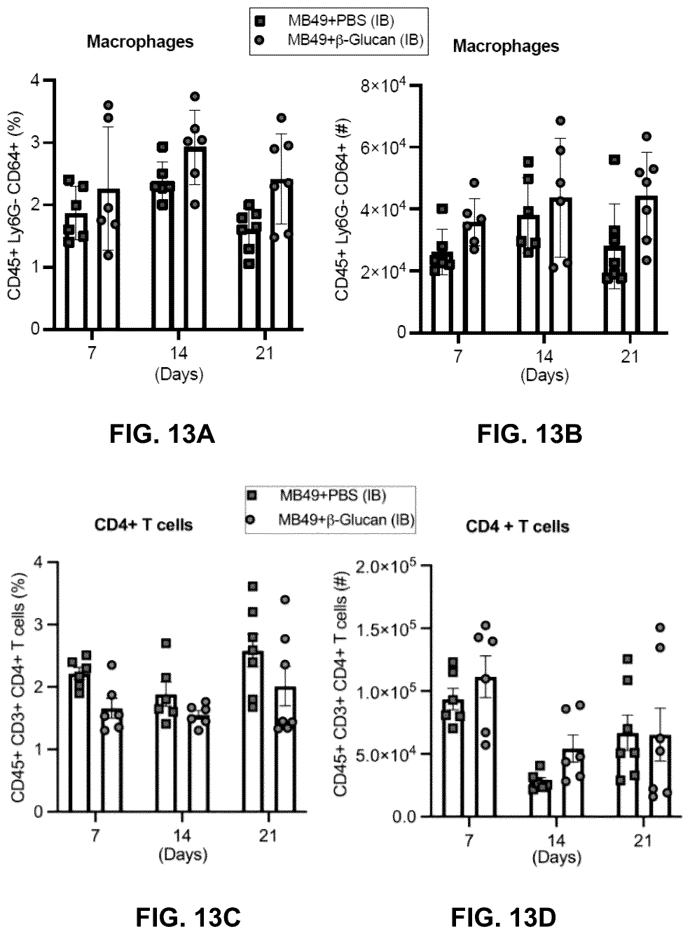

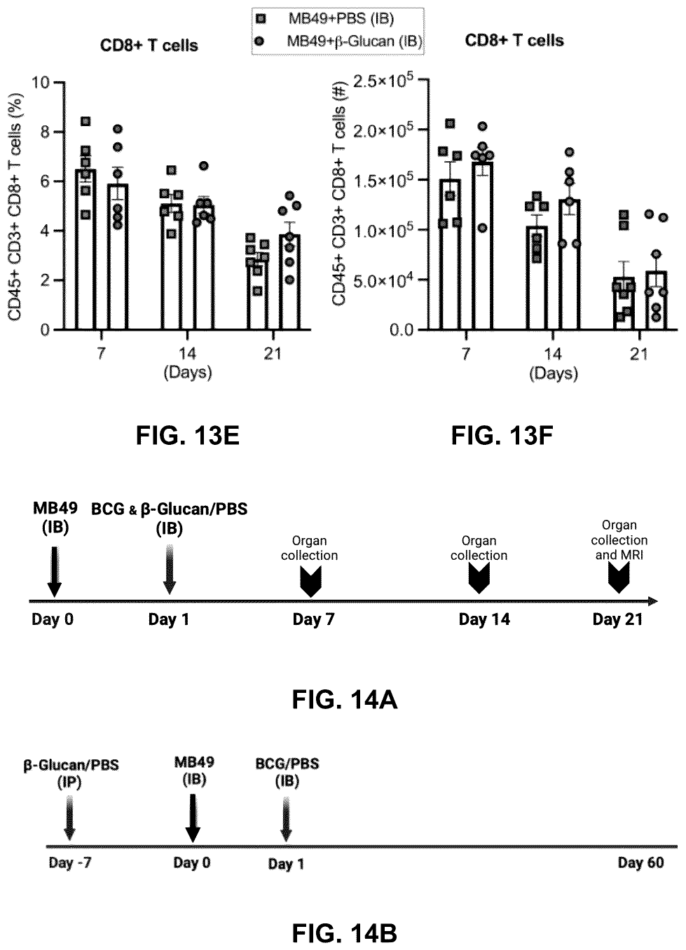

To evaluate the cellular immune response after β-glucan treatment in the bladder cancer model induction, a flow cytometry was conducted showing increased frequency and total cell number of neutrophils in the BM of β-glucan-IP treated mice (FIGS. 10A-10B). Since the β-glucan reprogramming of HSCs was biased towards granulopoiesis and neutrophils, the presence of neutrophils in the bladder was then assessed. After 6 days post-β-glucan treatment the frequency and absolute number of neutrophils (FIGS. 11A-11F and 12A-12B), but not macrophages (FIGS. 13A-13F), was significantly increased in the bladder. This indicates that the intravesical β-glucan treatment modulates the central trained immunity towards granulopoiesis and neutrophils, which are recruited into the bladder.

A Single Dose of Combined BCG and β-Glucan Intravesical Therapy Leads to Complete Tumor Clearance with 100% Survival

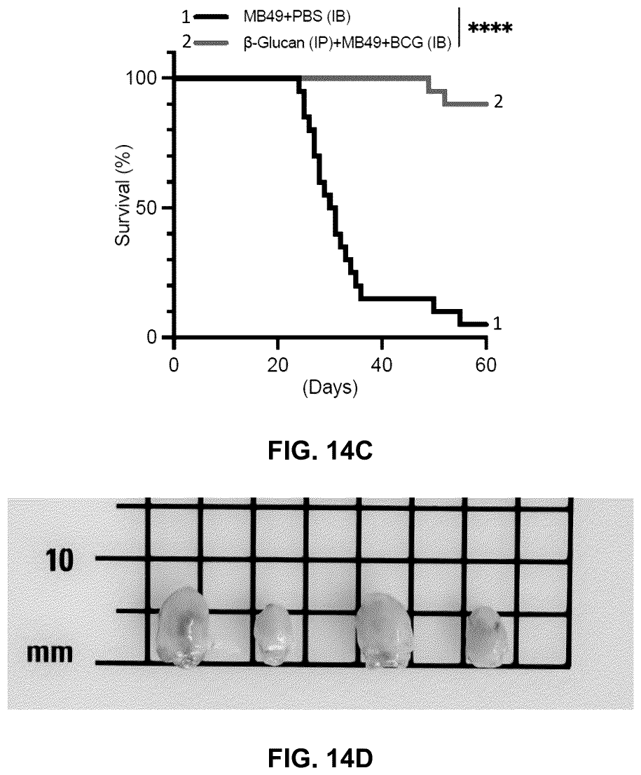

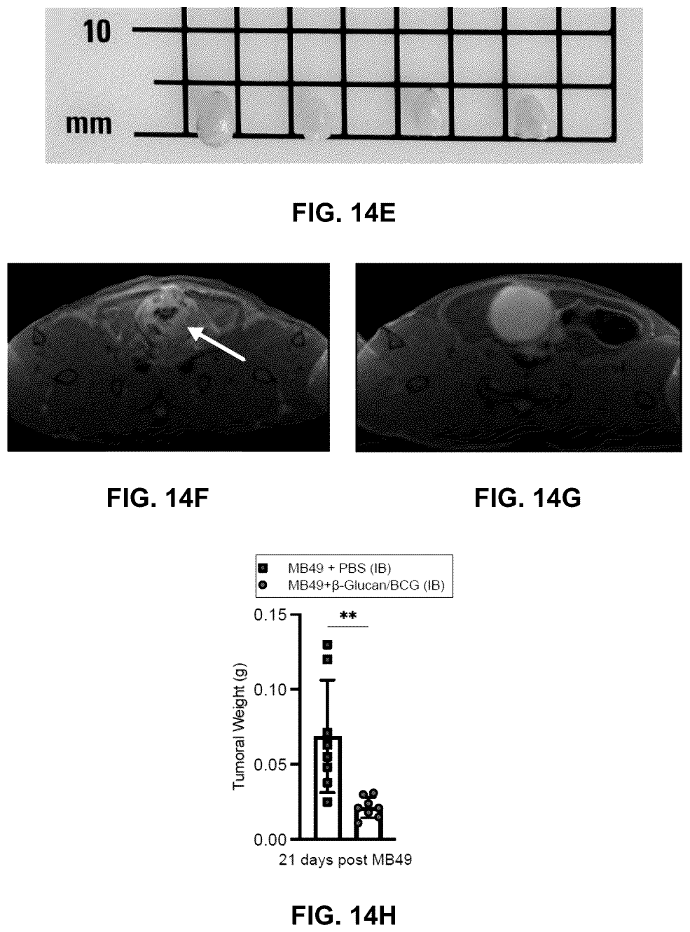

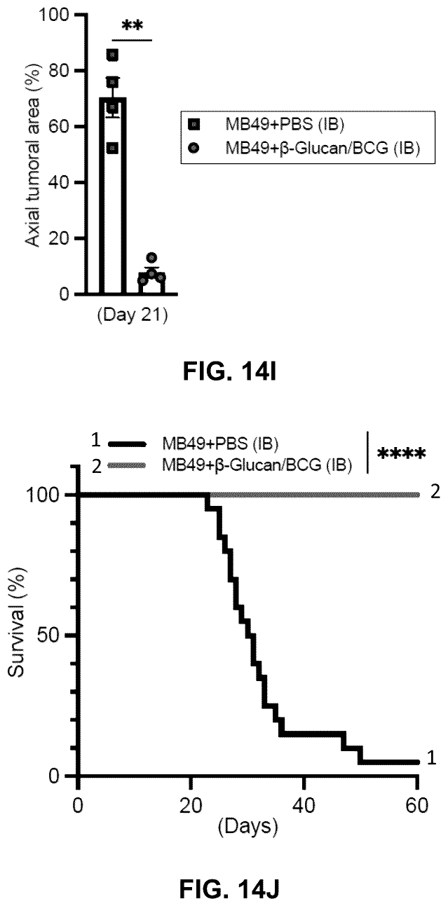

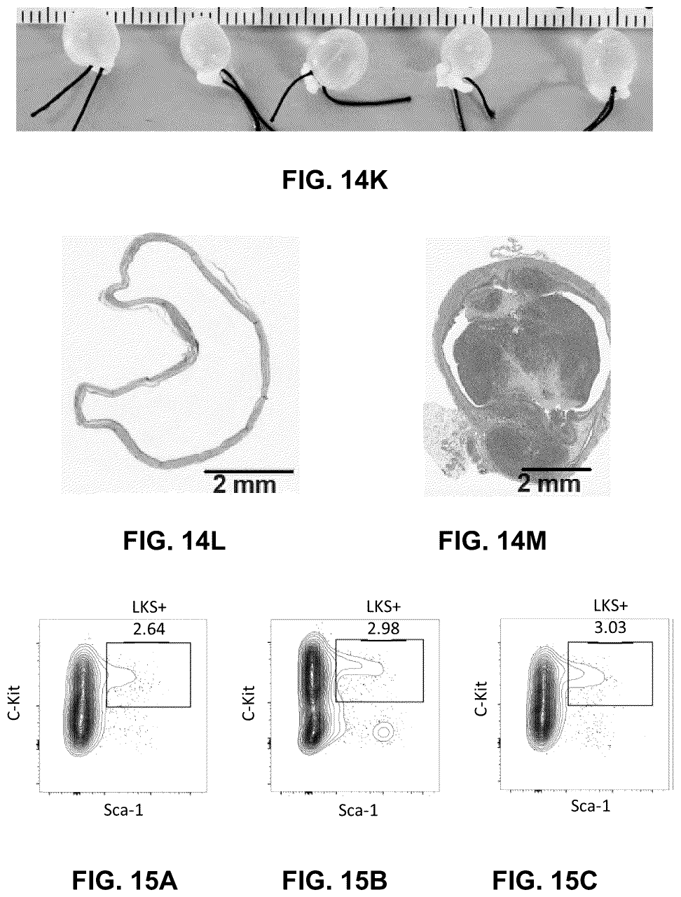

Considering that therapy with BCG or β-glucan individually led to reduced tumor growth and 50% increase in survival, it was then investigated whether the combination of both BCG/β-glucan can potentially enhance host antitumor immunity and subsequently survival (FIG. 14A). Initially, a single systemic dose of β-glucan (IP) was administered before the tumor implantation, followed by a single intravesical dose of BCG at day one post-tumor, resulting in a significant enhancement in the survival (90%) (FIGS. 14B-14C). However, it is important to note that clinically, bladder cancer patients receive BCG via intravesical route. Thus, it was subsequently investigated whether the addition of β-glucan to the intravesical BCG therapy can enhance the BCG anti-tumor effect. Surprisingly, administration of combined BCG with β-glucan in a single intravesical dose one day post-tumor implantation resulted in complete suppression of tumor growth as assessed by histopathology, MRI and tumoral weight (FIGS. 14D-14I) along with 100% survival (FIG. 14J). These animals were followed up to 80 days after the implantation of the tumor, and the bladders were harvested for macroscopic and histological examination, demonstrating the total clearance of the tumor in all the animals without any recurrence (FIG. 14K-14M).

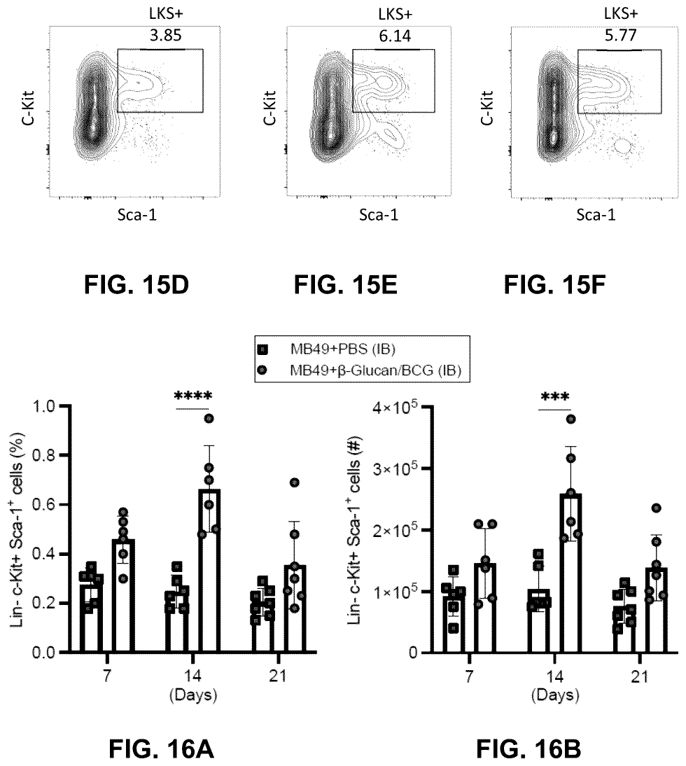

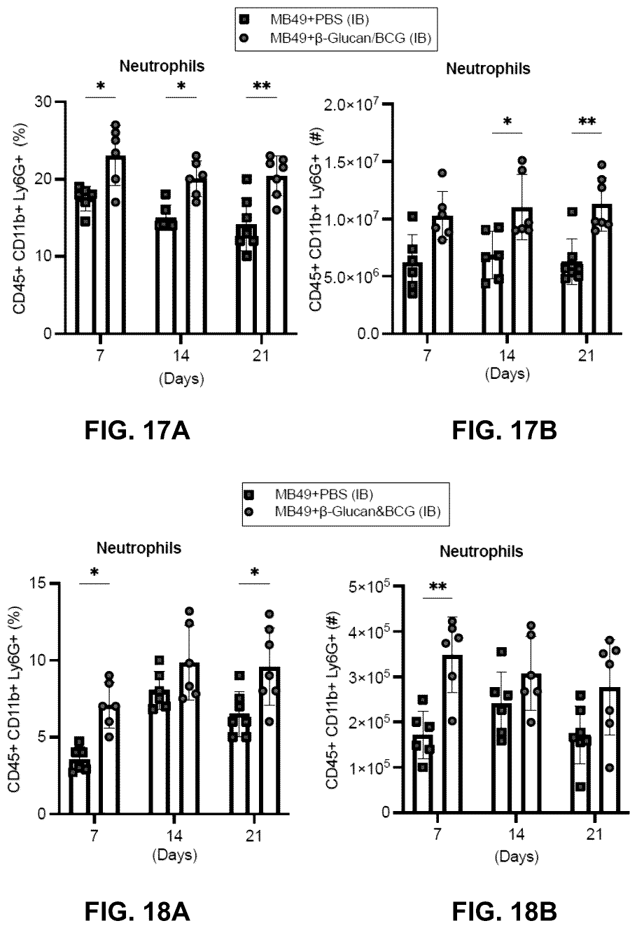

Immunophenotyping of the BM-cells revealed a significant expansion of the LKS⁺ cell population at day 14, similar to the effect observed with BCG-only treatment (FIGS. 4A-4B, 15A-15F and 16A-16B). It was also observed that the dual BCG/β-glucan intravesical therapy significantly increased the frequency and absolute number of neutrophils but not monocytes in all timepoints (day 7, 14, and 21) in the BM (FIGS. 17A-17B).

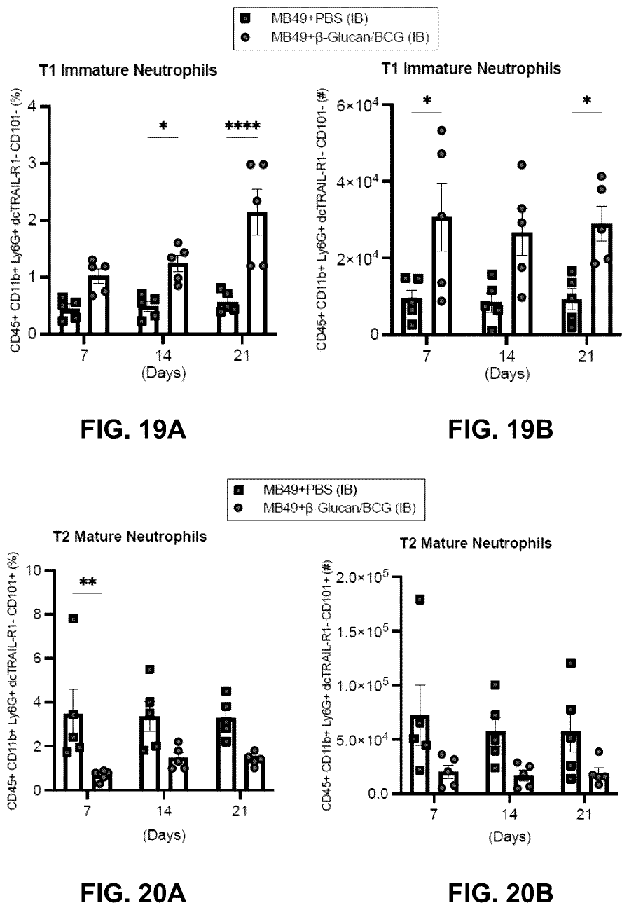

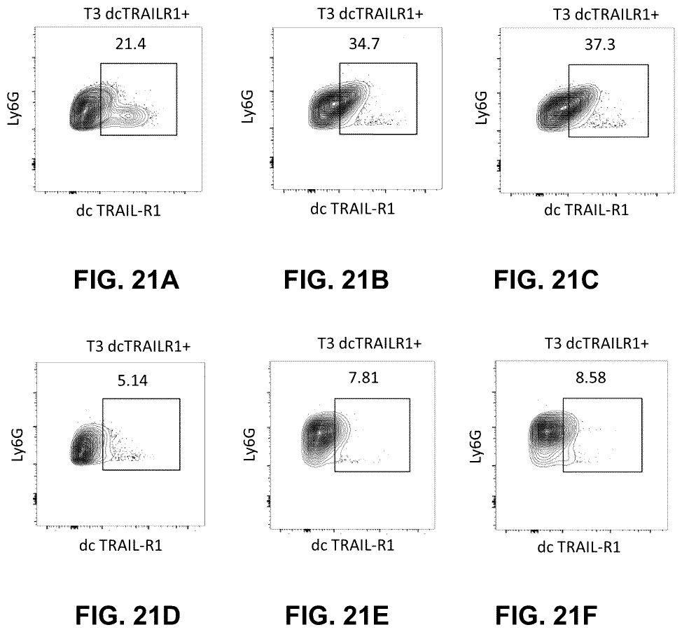

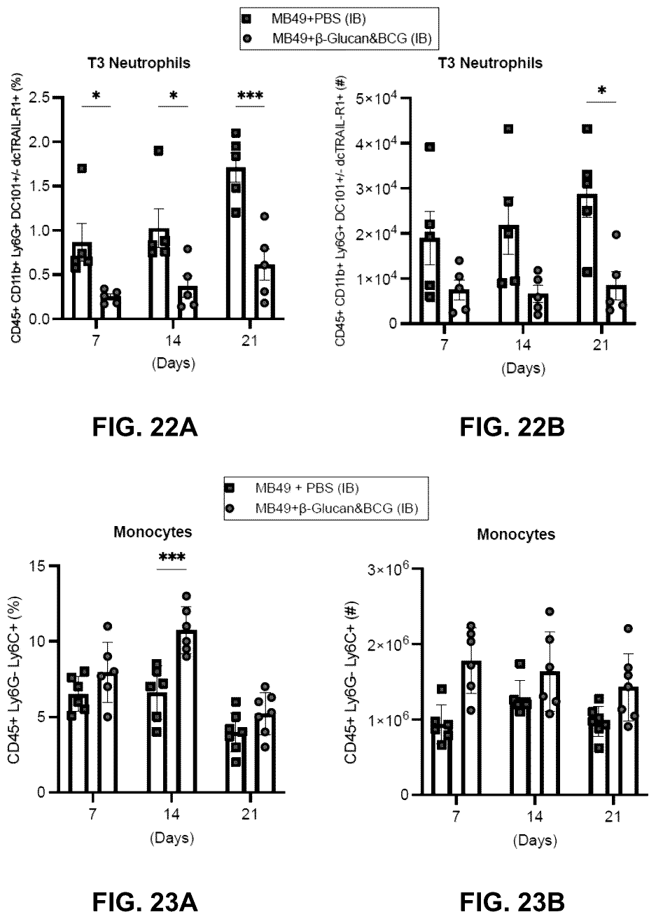

It has been shown that there is a diverse array of neutrophils varying in their maturation, surface markers expression, and distinct transcriptomic and epigenomic profiles. Intriguingly, there are three distinct neutrophil populations (T1, T2, and T3) within the tumor environment across mouse models and in multiple human cancers, with the T3 associated with a pro-tumor phenotype. It was also found that the frequency and total cell numbers of neutrophils in the bladder were increased after the dual BCG/β-glucan-IB (FIGS. 18A-18B). Importantly, the dual therapy trained neutrophils to maintain the T1 phenotype (FIGS. 19A-19B) which is known for its antitumoral properties and prevented their conversion to the T3 subset, which promotes tumor growth (FIGS. 20A-20B, 21A-21F, 22A-22B, and 23A-23B). Taken together, these findings indicate that the dual BCG/β-glucan intravesical therapy imprints a unique signature in HSCs and progenitor cells, biasing towards granulopoiesis and the generation of trained neutrophils that predominately retain their anticancer function within the tumor microenvironment. Interestingly, these trained neutrophils also exhibit resistance to conversion into T3 pro-tumoral neutrophils, a phenomenon regulated by the tumor microenvironment.

To investigate the specific mechanism underlying the antitumoral effects of trained neutrophils, the role of ROS in tumor inhibition was assessed using an in vitro co-culture system. Neutrophils were purified from the bone marrow of mice 14 days after IB treatment with BCG/β-Glucan or PBS IB and co-cultured with MB49 cells (ratio 1:1) in the presence or absence of the ROS scavenger N-Acetyl Cysteine (NAC). The neutrophils were purified from the BM using the EasySep Mouse Neutrophils Enrichment Kit following the manufacturer's instructions (Stem Cell Technology). Isolated cells were counted and washed (in cold sterile PBS). Purity was verified by flow cytometry and was always >75% neutrophils before transfer or coculture. 10⁶ neutrophils in 50 μL of PBS were transferred into tumor bearing mice via intravesical route. Mice were monitored for survival.

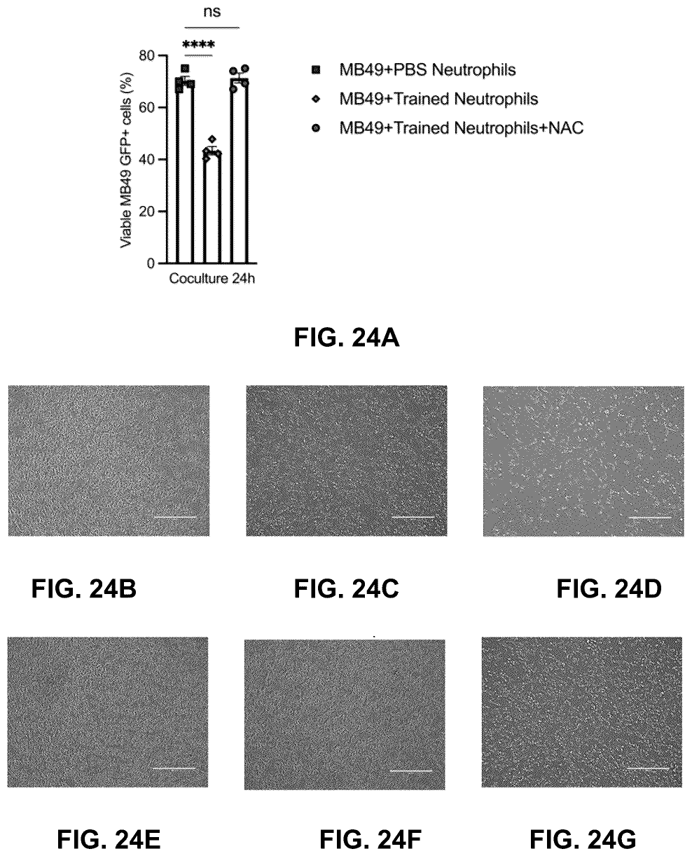

These experiments demonstrated that dual-trained neutrophils were remarkably effective in inducing tumor cell death, an effect that was ROS-dependent, as NAC presence abolished the cytotoxic activity of BCG/β-Glucan-trained neutrophils (FIGS. 24A-24G).

To better characterize neutrophil infiltration within the tumor microenvironment, an intravital microscopy (IVM) was developed with whole-mount imaging protocols for tumor-bearing bladders.

Bladder Intravital Microscopy A Nikon CSU-X1 multichannel spinning-disk confocal upright microscope was used to image mouse bladder. Briefly, mice were anaesthetized with 10 mg/kg xylazine hydrochloride and 200 mg/kg ketamine hydrochloride delivered intraperitoneal and their body temperature was maintained at 37° C. using a heating pad (World Precision Instruments). A tail vein catheter was placed in the animals to deliver antibodies of interest and to maintain anesthetic. Blood vessels in each mouse were labeled by injection of 100 μL Texas red-conjugated Dextran (70,000 MW; Invitrogen) at 1 mg/mL or CD31 antibody. The bladder was exposed by a 1 cm incision in the abdomen, oriented to visualize the tumour, and immobilized by using window and gentle suction.

Mice were intravenously administered with anti-Ly6G, anti-CD31, and anti-dcTRAIL-R1 antibodies (BioLegend) and euthanized after 1 hour (after IVM). Mice bladders were inflated with 100 μL 1.5% low melting point (LMP) agarose (Invitrogen) dissolved in Hank's Balanced Salt Solution (HBSS, Sigma-Aldrich). Bladders were dissected out and fixed with 4% Paraformaldehyde (PFA) overnight. Fixed bladders were embedded in 1.5% LMP agarose dissolved in HBSS and sectioned into 300 μm slices using a vibratome (Leica). Images were acquired on sections located from 1200 μm to 1800 μm using a Nikon CSUX-1 spinning disk confocal microscope.

Image analysis was conducted using FIJI (National Institute of Health) in a blinded manner. More specifically, Ly-6G channel was extracted, auto-contrasted and converted to 8-bit. Threshold was set as minimum 100 and maximum 255, and a selection was created to measure the size of Ly-6G+ area. dcTRAIL-R1 mean fluorescence intensity was calculated on Ly-6G+ area.

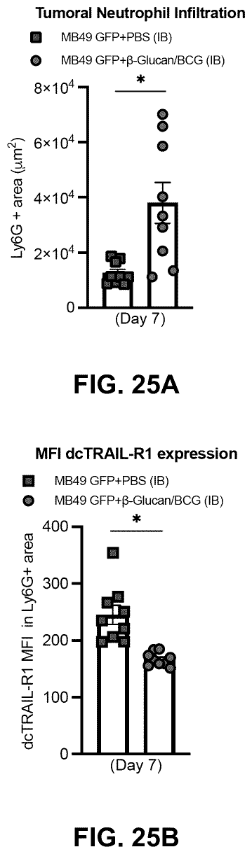

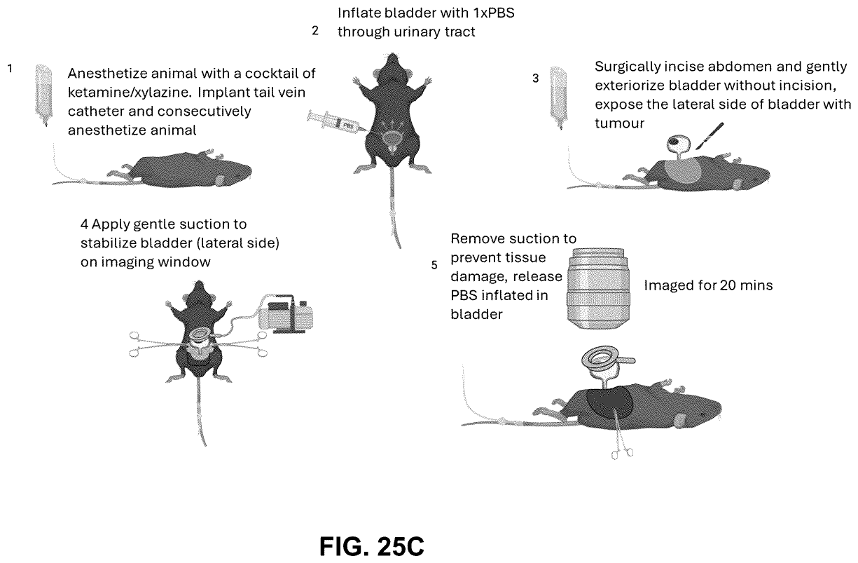

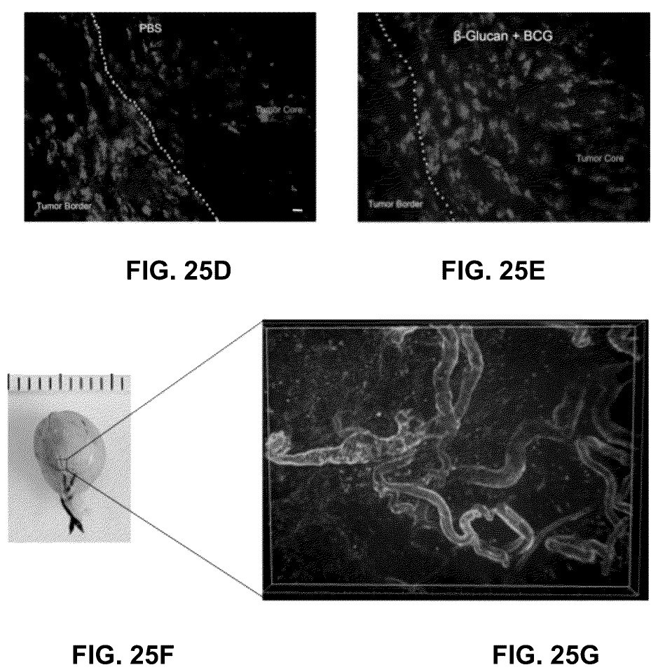

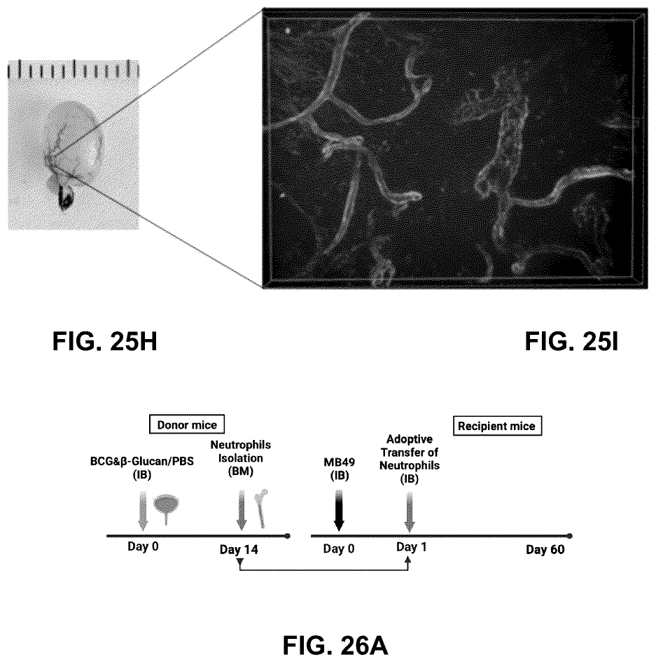

At days three and seven post-tumor instillation, dual-treated animals exhibited significantly increased neutrophil infiltration into the tumoral core (FIGS. 25A, 25C, 25D and 25E), alongside reduced expression of dcTRAIL-R1 a marker of pro-tumor (T3) neutrophils (FIG. 25B). In contrast, neutrophils in PBS-treated mice expressed high levels of dcTRAIL-R1 (FIG. 25B) and failed to infiltrate the tumor, instead accumulated at the tumor border. These IVM findings corroborate the above flow cytometry data. Additionally, 3D reconstruction of IVM-obtained videos revealed decreased neovascularization in the dual-treated group compared to PBS-treated animals (FIGS. 25F-251).

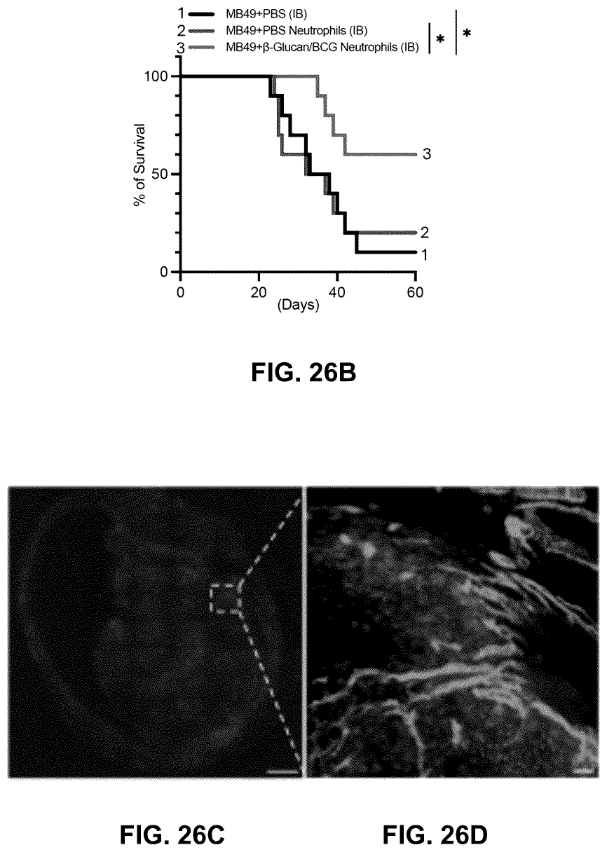

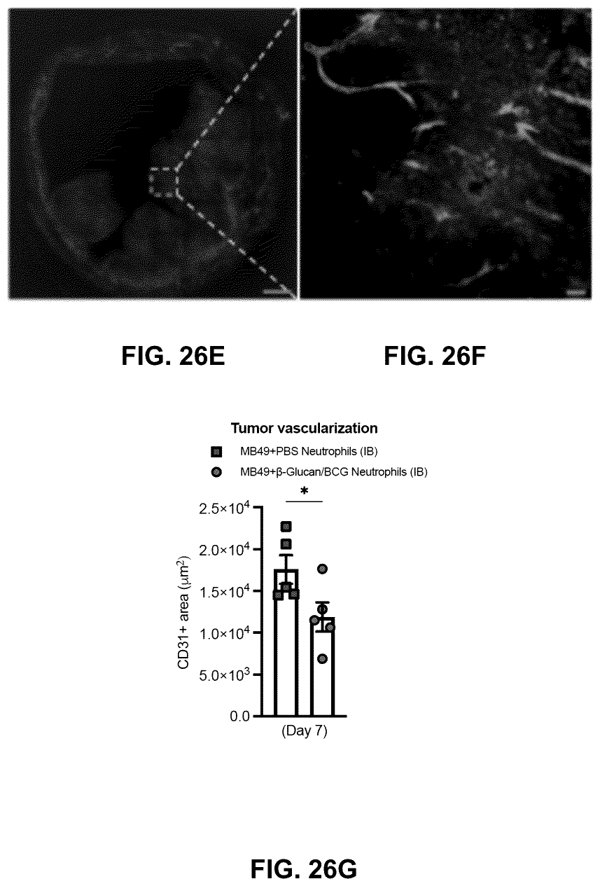

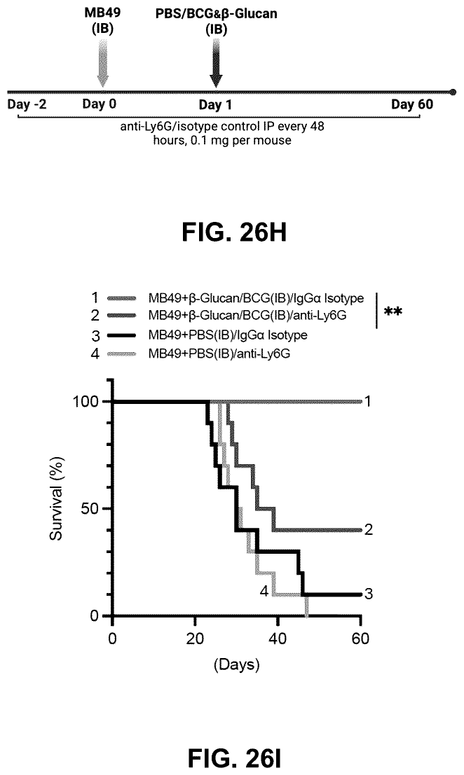

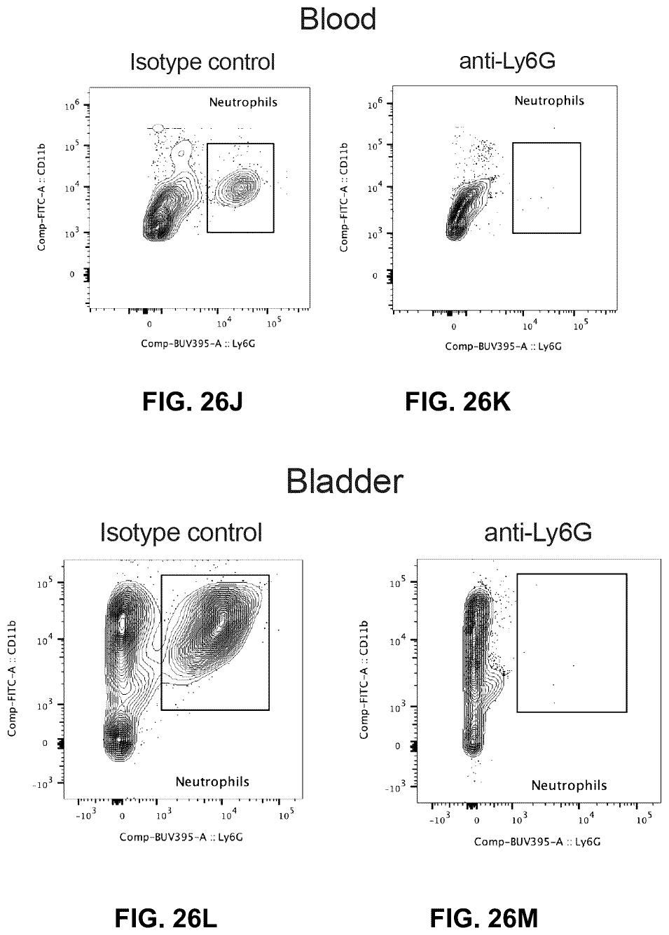

To further demonstrate the role of BCG/β-Glucan trained neutrophils in tumor control, adoptive intravesical transfer of neutrophils was performed into tumor-bearing mice (FIG. 26A). Survival was significantly improved in mice that received BCG/β-Glucan-trained neutrophils compared to those receiving PBS-treated neutrophils (FIG. 26B). This effect appears to be partially mediated by the ability of trained neutrophils to impair tumor neovascularization, as demonstrated by a marked reduction in tumoral vascularization following IB instillation BCG/β-Glucan-trained neutrophils (FIGS. 26C-26G). To further validate the role of neutrophils in tumor suppression, a loss of function experiment was conducted by depleting neutrophils in tumor-bearing mice treated with either BCG/β-Glucan or PBS. Neutrophil depletion abolished the therapeutic benefit of the dual intravesical therapy, reducing survival from 100% to 40% (FIGS. 26H-26M). These findings underscore the essential role of trained neutrophils in mediating tumor suppression and enhancing survival, providing strong mechanistic evidence for their contribution to BCG/β-Glucan mediated antitumor immunity.

Altogether the present data suggest that BCG/β-Glucan enhances the antitumoral capacity of neutrophils through two mechanisms. First, it induces a state of heightened responsiveness associated with trained granulopoiesis, characterized by enhanced ROS production by neutrophils. Second, it educates the neutrophils to resist conversion into a protumoral phenotype driven by the tumor microenvironment impairing angiogenesis and subsequently tumoral growth. The observed increase in survival underscores the potential of BCG/β-Glucan-trained neutrophils to mount a more robust immune response against tumor cells within the bladder. Overall, these results highlight the translational relevance of leveraging trained immunity in neutrophils as a novel strategy to enhance the intravesical immunotherapy for bladder cancer.

### Antitumoral Effects of BCG/β-Glucan Across Other Solid Tumors

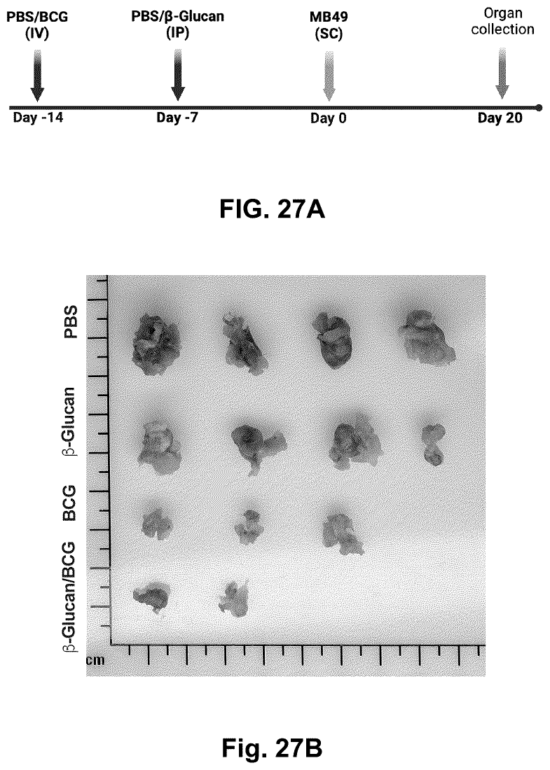

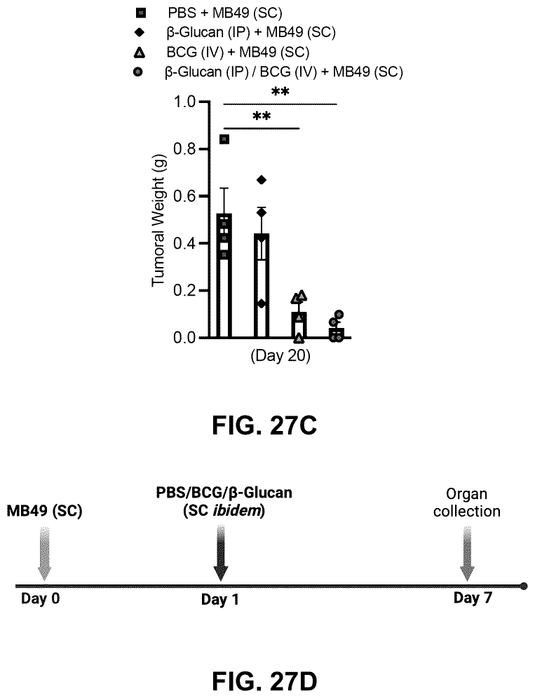

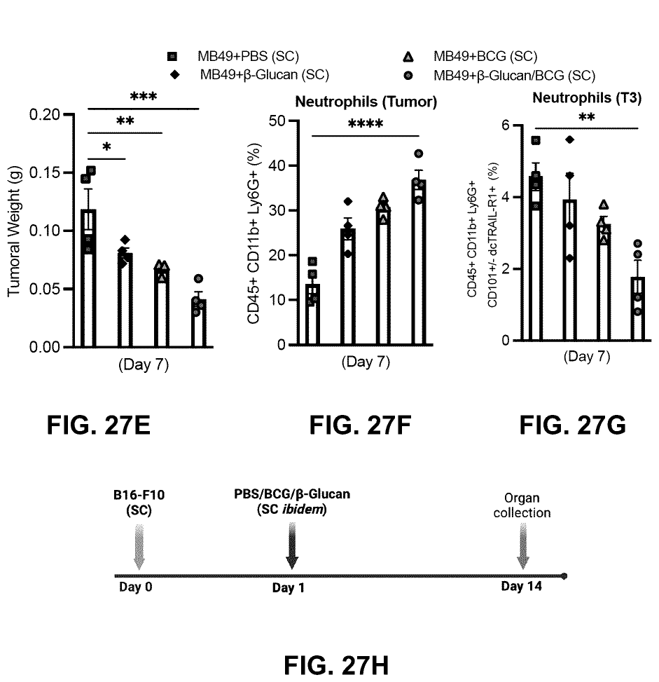

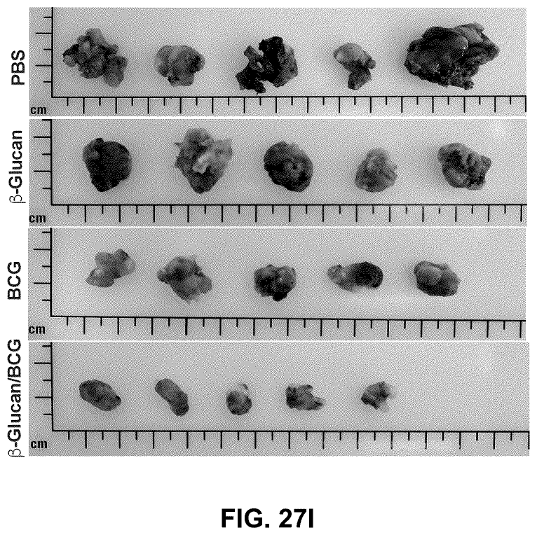

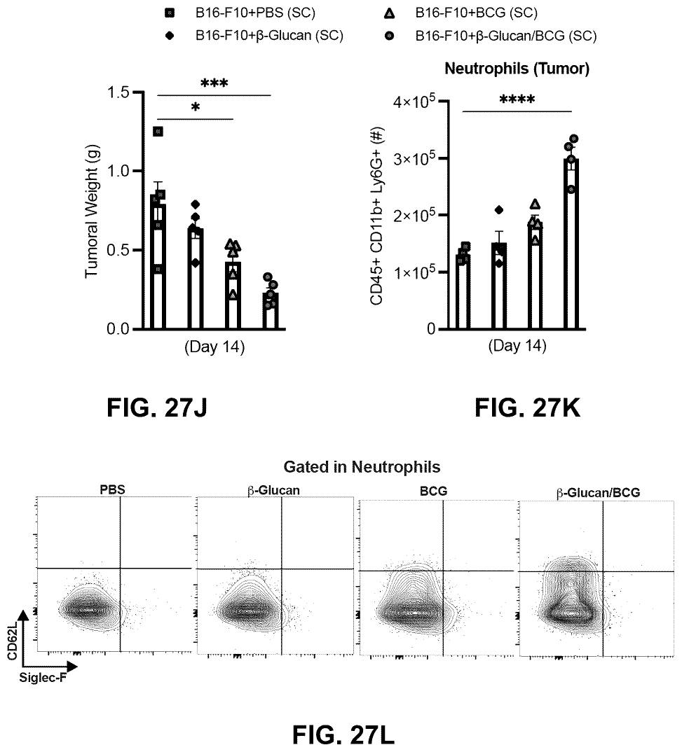

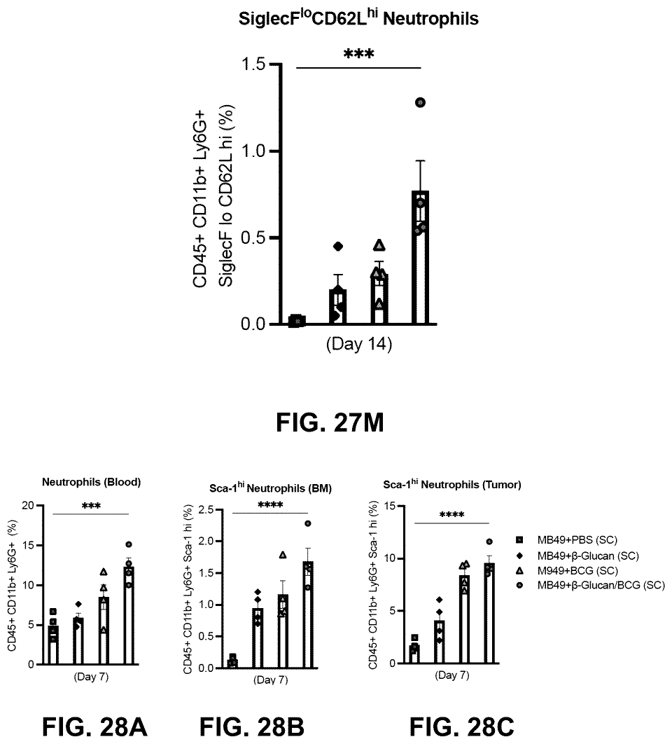

Given the remarkable antitumoral effect of the dual intravesical therapy with BCG/β-Glucan in the preclinical bladder cancer model, as well as its unique ability to train neutrophils, it was next investigated whether this therapeutic benefit can be extended to other solid tumors. A focus was placed on melanoma, a cancer with poor clinical prognosis and limited treatment options, for which early clinical experience has shown a successful response to BCG immunotherapy in humans. To assess this, initially a systemic dose of BCG, β-Glucan, or their combination was administered to mice before challenging them with subcutaneous MB49 and monitored tumor progression over 20 days (FIG. 27A). Interestingly, while all PBS- and β-Glucan-treated animals developed tumors, those pre-treated with BCG showed significantly reduced tumor growth. More importantly, animals receiving the combination treatment developed the smallest tumors, with complete tumor regression observed in two cases after 20 days of tumor challenge (FIGS. 27B-27C). These findings highlight the synergistic antitumoral effect of combining BCG and β-Glucan, suggesting the adjuvant role of 3-Glucan in enhancing BCG-mediated tumor suppression through the induction of trained immunity.

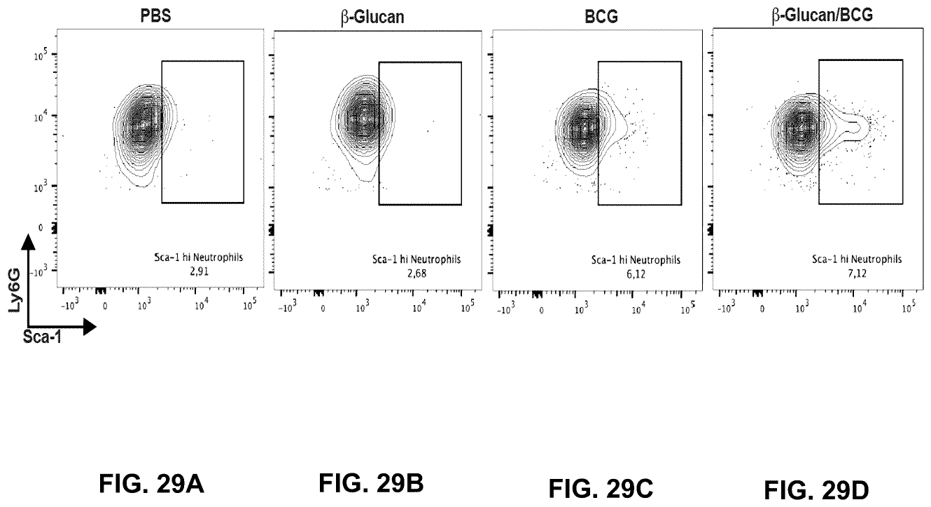

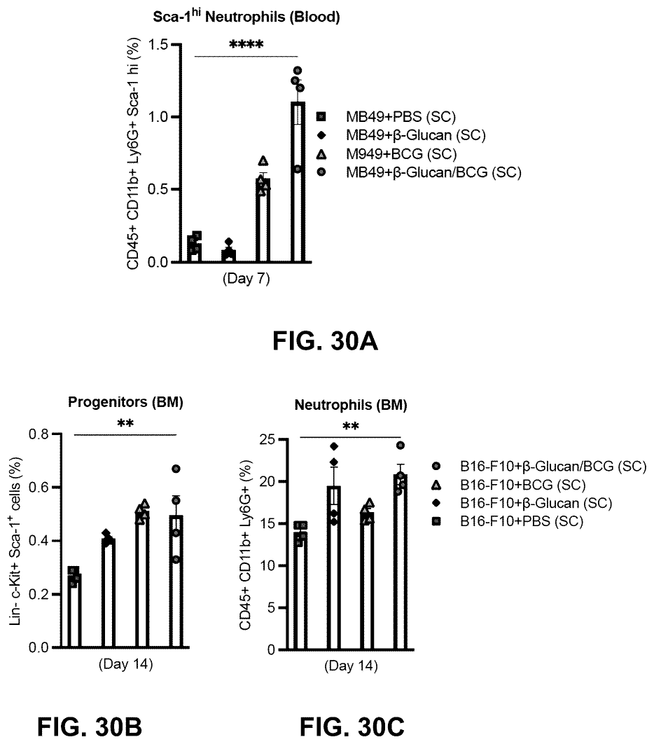

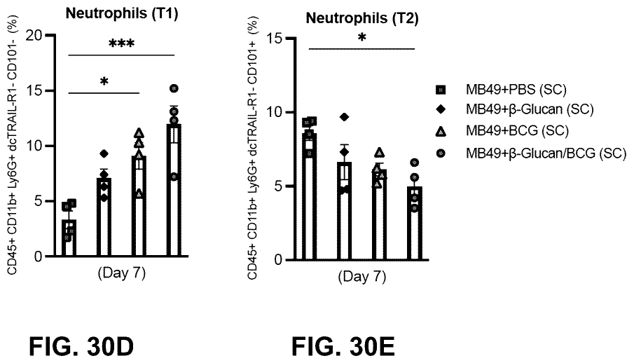

Next, the potential antitumoral effects of all treatment conditions was investigated in a therapeutic setting (post-tumor induction). Animals were subcutaneously injected with either MB49 bladder cancer cells (FIG. 27D) or B16-F10 melanoma cells (FIG. 27H) and treated one day after with a single dose of β-Glucan, BCG, or the combination at the same site where the cancer cells were implanted. Tumor progression was monitored for up to 14 days, after which tumors, bone marrow, and blood were collected for immunophenotyping. Consistently, across all treatment conditions, BCG/β-Glucan dual therapy induced the most significant reduction in tumor growth in both subcutaneous bladder cancer (FIG. 27E) and melanoma models (FIGS. 27I-J, 29A-29D and 30A). This effect was accompanied by an increase in neutrophils in the bone marrow, blood and tumors, observed in both MB49 (FIGS. 27F and 28A-28C) and melanoma (FIGS. 27K and 30B-30C). Importantly, a significant expansion of the BM-LKS⁺ population was also observed in melanoma-bearing animals treated with BCG or the dual therapy (FIGS. 30B-30C). Notably, a marked reduction in protumoral neutrophils (T3) was confirmed in dual-treated of MB49 tumor-bearing animals (FIGS. 27G and 30D-30E), along with the induction of an interferon-responsive phenotype (Siglec-F^{io}, CD62L^{hi} and Sca-1^{hi}) in the BM, circulating, and tumor-associated neutrophils in both MB49 (FIGS. 28A-28C, 29A-29D and 30A-30E) and melanoma models (FIGS. 27L and 27M). This interferon-responsive phenotype is considered a hallmark of successful immunotherapy, aligning with the antitumoral phenotype initially described in the orthotopic bladder cancer model.

Collectively, the BCG/β-Glucan therapy demonstrated superior antitumoral effects in both orthotopic bladder cancer and melanoma models, while uniquely training neutrophils to enhance their antitumoral capacity. These findings suggest that this therapeutic approach could have broad clinical applicability, potentially improving the antitumoral immune response in other solid tumor types.

### Discussion of the Experimental Results

The utilization of live bacteria or bacterial toxins (e.g. Streptococcus pyogenes) in cancer therapy dates back to the 19^{th} century, with treatments targeting solid tumors demonstrating mixed clinical outcomes and unclear mechanism of action. Similarly, BCG has been used as a treatment against bladder cancer for more than 50 years, yet the precise mechanisms of action of BCG immunotherapy remained incompletely understood.

The advent of immunotherapy has revolutionized the successful treatment for various types of cancer. For instance, the discovery of checkpoint inhibitors, such as PD-1, for T cell-mediated immunity, represented a significant therapeutic breakthrough in cancer treatment. Indeed, the therapeutic success of BCG in bladder cancer is partially attributed to T cell-mediated immunity with repeated BCG installations required for a robust T cell infiltration into the bladder. It was also recently demonstrated that BCG-IB treatment reprograms HSCs and generates trained immunity with anti-tumor capacity in both murine models and patients with bladder cancer, providing a mechanistic insight into BCG immunotherapy.

However, despite the activation of innate and adaptive immunity, the efficacy of BCG treatment in bladder cancer is between 30-50% with up to 40% of patients experiencing relapse. Therefore, the present experiments aimed to investigate strategies to enhance the antitumoral capacity of BCG in bladder cancer via improving its efficacy and reducing the number of repeated installations.

Given the evolutionary timeline of vertebrates and the success of plants and invertebrates which depend solely on innate immunity, immune memory cannot be considered an innovation of only the lymphoid lineage. Indeed, the evolutionary innate immune memory program is a conserved mechanism whereby innate immune cells can induce a heightened response to a secondary stimulus due to metabolic and epigenetic reprogramming. Importantly, the longevity of this memory phenotype can be attributed to the reprogramming of self-renewing hematopoietic stem cells (HSCs) in the bone marrow, which is subsequently transmitted to lineage-committed innate immune cells.

The present example investigated the impact of β-glucan or BCG independently as well as the dual BCG/β-glucan therapy in an orthotopic bladder tumor model. Repeated intravesical BCG treatment not only promoted both HSC-mediated trained immunity but also elicited an adaptive immune response, resulting in significant tumor reduction and 50% survival (FIGS. 1A-1L, 2A-2F, 3A-3B, 4A-4G, 5A-5G, 6A-6H, 7A-7B, and 8A-8L). Similarly, treatment with β-glucan also led to a significant reduction in tumor volume and 40% increase in survival (FIGS. 9A-9L, 10A-10B, 11A-11F, 12A-12B, and 13A-13F). This protection was primarily mediated through trained immunity, as the frequency of adaptive immune cells remained unchanged following β-glucan IB treatment. Unexpectedly, a single dose of intravesical dual BCG/β-glucan treatment resulted in complete tumor elimination in the bladder and 100% survival with no relapse (FIGS. 14A-14M, 15A-15F, and 16A-16B). These findings are particularly remarkable considering the aggressive nature of MB49 cancer cells and underscore the potential of translating BCG/β-glucan therapy into a clinical trial.

A rapid recruitment of a large number of neutrophils from circulation into the infected or damaged tissues is a unique feature, which has been hijacked by cancer to promote tumor growth. Both immature and mature neutrophils infiltrating the tumor and differentiate into transitional T1 and T2 populations. However, the tumor microenvironment subsequently induce a terminal differentiation into the T3 pro-tumor neutrophils, which is a predictor of poorer prognosis in patients with solid tumors.

Flow cytometry analysis of all three conditions (BCG, β-glucan, and BCG/β-glucan) revealed a shared signature of enhanced granulopoiesis and significant mobilization of neutrophils to the bladder. Consequently, it appears that the primary impact of BCG and β-glucan is to promote trained immunity. The present example demonstrated that the combination therapy of BCG/β-glucan promoted a unique subset of neutrophils that maintain their anticancer phenotype by resisting conversion into T3 pro-tumor neutrophils (FIGS. 17A-17B, 18A-18B, 19A-19B, 20A-20B, 21A-21F, 22A-22B, 23A-23B, 24A-24G, and 25A-251). Furthermore, the therapeutic approach and molecular mechanism demonstrated with bladder cancer translated into another solid tumor, melanoma (FIGS. 27A-27M). The sustained anti-tumor capacity of BCG/β-glucan-trained neutrophils during early stages of tumor growth was sufficient for tumor elimination without the involvement of T cells. This stands in contrast with BCG treatment, which necessitates repeated administration for the optimal engagement of T cell-mediated immunity in bladder cancer.

It is presently demonstrated that the addition of β-glucan to the BCG treatment in bladder cancer can significantly enhance the efficacy of BCG and reduced the number of BCG installations. As β-glucan has been used in different human clinical trials, it can be safely used with BCG for treating patients with bladder cancer.

What is claimed is:

1. A method of treating cancer in a subject in need thereof, the method comprising administering a therapeutically effective amount of Bacillus Calmette-Guérin (BCG) vaccine and β-glucan adjuvant, wherein the cancer is characterized by the presence of protumoral T3 neutrophils in the tumour microenvironment, and wherein the β-glucan has a β-1,3 glucose backbone.

2. The method of claim 1, wherein the β-1,3 backbone of the β-glucan has β-1,6 side branching.

3. The method of claim 2, wherein the β-glucan is derived from fungus or yeast.

4. The method of claim 1, wherein the β-glucan has a molecular weight of from 100 kDa to 900 kDa.

5. The method of claim 1, wherein the cancer is bladder cancer, melanoma, lung adenocarcinoma, head and neck squamous cell cancer, pancreatic adenocarcinoma, low-grade gliomas, esophageal carcinoma, or cervical squamous cell carcinoma.

6. The method of claim 5, wherein the cancer is bladder cancer or melanoma.

7. The method of claim 6, wherein the bladder cancer is non-muscular invasive bladder cancer.

8. The method of claim 1, wherein the BCG vaccine comprises an attenuated strain of Mycobacterium bovis which is a TICE strain, Frappier strain, Danish strain 1331, Glaxo 1077 strain, Tokyo 172-1 strain, Pasteur 1173 P2 strain, Moscow-I strain, RIVM strain, Connaught strain, Russia strain or Moreau strain.
